(19) United States
(12) Patent Application Publication
(10) Pub. No.: US 2023/0338297 A1
(43) Pub. Date: Oct. 26, 2023

(54) Encapsulated Agents that Bind to MCT-1

(71) Applicant: THE JOHNS HOPKINS UNIVERSITY, Baltimore, MD (US)

(72) Inventors: Jordina RINCON-TORROELLA, Baltimore, MD (US); Bert Vogelstein, Baltimore, MD (US); Kenneth W. Kinzler, Baltimore, MD (US); Shibin Zhou, Baltimore, MD (US); Nickolas Papadopoulos, Baltimore, MD (US); Marco Dal Molin, Baltimore, MD (US); Surojit SUR, Baltimore, MD (US)

(21) Appl. No.: 18/024,293

(22) PCT Filed: Aug. 31, 2021
(86) PCT No.: PCT/US2021/048372

§ 371 Date: Mar. 2, 2023

Related U.S. Application Data
(60) Provisional application No. 63/075,758, filed on Sep. 8, 2020.

(51) Int. Cl. A61K 9/50; A61P 35/00; A61K 45/06; G01N 33/50; A61K 31/19

(52) U.S. Cl. CPC A61K 9/5036; A61P 35/00; A61K 45/06; G01N 33/5017; A61K 31/19

(57) ABSTRACT

The present disclosure provides compositions comprising at least one cyclodextrin and at least one cytotoxic receptor binding small-molecule. Also disclosed are kits containing said compositions. The compositions of the present disclosure can be administered to a subject suffering from at least one type of cancer.

### CROSS-REFERENCE TO RELATED APPLICATIONS

This application is a national stage application of PCT/US2021/048372, filed Aug. 31, 2021, which claims priority to U.S. Application No. 63/075,758 filed on Sep. 8, 2020, the contents of which are herein incorporated by reference.

### GOVERNMENT SUPPORT INFORMATION

This invention was made with government support under grant CA062924 and 5R25NS065729 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,354 Byte ASCII (Text) file named “38697_252_ST25.TXT,” created on Mar. 1, 2023.

### FIELD

The present disclosure relates to compositions comprising at least one cyclodextrin and at least one cytotoxic receptor binding small-molecule and kits containing said compositions. The compositions of the present disclosure can be administered to a subject suffering from at least one type of cancer.

### BACKGROUND

Tumors are metabolic entities that comprise cancer and host cells. Their metabolic activities depend on access to nutrients, biological activities, and spatiotemporal localization. Payen et al., Molecular Metabolism, 33:48-66 (2020). While most cells in the body are oxidative and fully oxidize glucose to CO₂, cells exposed to hypoxia and proliferating cells, preferentially convert glucose to lactate in processes known as anaerobic and aerobic glycolysis. Id. These metabolic phenotypes are at the core of tumor biology. In solid tumors, the glycolytic switches associated with adaptation to hypoxia and cell proliferation operate via different mechanisms. Id. In fact, at the whole tumor level, increased conversion of glucose to lactate associated with a high glycolytic rate generates millimolar concentrations of lactic acid that is released to the extracellular compartment. Id. Because lactic acid is hydrophilic and a weak acid, its transport across membranes necessitates transporters that belong to the monocarboxylate transporter (MCT) family.

MCTs are encoded by the solute carrier 16 (SLC16) family of genes. There are currently 14 members of the family, including MCT-1/SLC16A1, MCT-2/SLC16A7, MCT-3/SLC16A8, and MCT-4/SLC16A3. Structurally, they comprise 12 transmembrane (TM) helices, intracellular N- and C-termini and a large cytosolic loop between TM6 and TM7. Id.

While MCTs share common substrates, including pyruvate, L-lactate, ketone bodies aceto-acetate and D-b-hydroxybutyrate, and short chain fatty acid propionate and butyrate, they differ by their relative affinities. MCT-2/SLC16A7is the transporter with the highest affinity for mono-carboxylates, followed by MCT-1/SLC16A1, MCT-3/SLC16A8 (which has an affinity for lactate comparable to MCT-1), and MCT-4/SLC16A3 that has a low affinity for lactate). Id. Although lactate is not the only substrate of MCTs, it is the most characterized in the literature and the most abundant in vivo, particularly in tumors where it reaches concentrations up to 40 mM. Id.

MCT-1, MCT-2, and MCT-4 expression has been extensively characterized in cancer cell lines and in multiple tumor types from patients. Upregulation of MCT-1, MCT-2, and MCT-4 during tumor progression from normal to tumor epithelium has also been repeatedly observed in human samples. Id. In fact, the upregulation of MCI-1 expression observed in many cancers has generated interest in targeting this protein for cancer treatment. To date, several inhibitors of MCT-1 have been reported that appear to have promising activity in experimental cancer models, including SR13800 and AZD3965. However, preclinical data shows limited efficacy, and preliminary clinical trial results reported potentially drug related adverse effects. Therefore, there is a need in the art alternative cancer treatments that target (e.g., bind to) MCT-1 that are safe and efficacious.

### SUMMARY

In one aspect, the present disclosure provides a composition comprising at least one cyclodextrin, such as a β-cyclodextrin, and at least one cytotoxic receptor binding small-molecule, where the β-cyclodextrin encapsulates the cytotoxic receptor binding small-molecule.

In some aspects, at least one α-D-glucopyranoside unit of the cyclodextrin in the composition has at least one hydroxyl chemical group replaced with an ionizable chemical group resulting in a negative charge and wherein the cyclodextrin encapsulates the at least one cytotoxic receptor binding small-molecule. In another aspect, at least one C2, C3, and C6 hydroxyl chemical groups of at least one α-D-glucopyranoside unit of the cyclodextrin are replaced with ionizable chemical groups. In other aspects, at least one α-D-glucopyranoside unit of the cyclodextrin is selected from the group consisting of two, three, four, five, six, seven, eight, and all α-D-glucopyranoside units of the cyclodextrin. In still further aspects in the composition, the ionizable chemical group is (i) the same at all replaced positions; and/or (ii) a weakly basic functional group or a weakly acidic functional group. In still further aspects, the weakly basic or weakly acidic functional groups are selected from the group consisting of amino, ethylene diamino, dimethyl ethylene diamino, dimethyl anilino, dimethyl naphthylamino, succinyl, carboxyl, sulfonyl, and sulphate functional groups.

In still further aspects, the above-described composition can be a liquid or solid pharmaceutical formulation.

In yet other aspects, when the cyclodextrin is a β-cyclodextrin, the β-cyclodextrin is selected from the group consisting of 6′ modified β-cyclodextrin, 6′ mono-succinyl β-cyclodextrin, hydroxypropyl-β-cyclodextrin, and succinyl-β-cyclodextrin.

In yet still other aspects, the cytotoxic receptor-binding small molecule used in the composition is a haloacetate, halopyruvate, halolactate, halopropionate or halobutyrate or combinations thereof.

In still further aspects, the above-described composition is formulated for systemic administration.

In another aspect, the present disclosure provides a kit comprising the above-described composition and instructions for use.

In yet another aspect, the present disclosure provides a method of treating a subject having a cancer. The method comprises administering to the subject a therapeutically effective amount of the above-described composition. In this method, the composition can be administered systemically (such as, for example, orally, intravenously, intrathecal, intraperitoneally, subcutaneously, and by intramuscular administration).

The above-described method can further comprise the step of administering at least one additional anti-cancer therapy. This additional anti-cancer therapy can be administered, simultaneously or sequentially, before or after above-described composition is administered.

In some aspects, the method involves treating a subject for a cancer expressing MCT-1. In other aspects, the cancer is selected from the group consisting of liver cancer, pancreatic cancer, bile duct cancer, colorectal cancer, mesothelioma, leukemias, germ cell tumors, glioma, lung cancer, ovarian cancer, prostate cancer, head and neck cancers, melanoma, stomach cancer, bone cancer, renal cancer, bladder cancer, and breast cancer.

In some aspects, when the cancer is breast cancer, the breast cancer is triple negative breast cancer.

In yet other aspect, the subject being treated is a mammal, such as a human.

In still further aspects, the present disclosure provides a method for assessing the stability of a composition comprising at least one agent encapsulated in a cyclodextrin (such as a β-cyclodextrin). Specifically, the method comprises the steps of:
a. providing at least one cyclodextrin (such as a β-cyclodextrin) and at least one agent, wherein the at least one agent is encapsulated in the at least one cyclodextrin to provide at least one cyclodextrin encapsulated agent composition;
b. assessing the composition for presence or absence of 3-bromopyruvate (3BP) using a cell toxicity assay; and
c. determining the stability of the composition.

In one aspect in the above-described method, the composition is incubated in sera for at least 30 minutes prior to performing the cell toxicity assay.

In another aspect in the above-described method, the agent is at least one cytotoxic receptor binding small-molecule.

Other aspects and embodiments of the disclosure will be apparent in light of the following detailed description and accompanying figures.

### DESCRIPTION OF THE FIGURES

FIG. 1 shows the encapsulation of different microencapsulated β-cyclodextrin complexes using size-exclusion chromatography (SEC) as described in Example 1. The complexes examined were: i) free 3BP (1 mg/mL); ii) succinyl-β-CD (20 mg/mL); iii) a mixture of 10 μL of free 3BP and 10 μL succinyl-β-CD; and iv) CD-3BP (10 mg/mL). Samples were monitored at 220 nm as shown in FIGS. 1A-1D. Specifically, free 3BP (1 mg/mL) is shown in FIG. 1A, succinyl-β-CD (20 mg/mL) is shown in FIG. 1B, a mixture of 10 μl of 3BP and 10 μl of succinyl-β-CD is shown in FIG. 10, and CD-3BP (10 mg/mL) in FIG. 1D.

FIG. 2 shows the rapid degradation of free 3BP in sera while β-cyclodextrin microencapsulated versions offered different degrees of protection against such degradation as described in Example 1. Specifically, the sCD-3BP complex appeared to be the most stable under these conditions for up to 8 hours and maintained near complete levels of activity at various concentrations while HPCD-3BP complex offered intermediate level of protection with significant loss of activity seen after 2 hours.

FIG. 3 demonstrates the potent cell death of sCD-3BP on various pancreatic cell lines as described in Example 2. Specifically, to demonstrate broad activity on pancreatic cancer, the cell lines were treated with varying concentrations of sCD-3BP ranging from 0 to 220 uM. All cell lines, except for CFPAC-1, exhibited exquisite sensitivity sCD-3BP, with their IC50s ranging from 27-33 uM as shown in FIG. 3A and 3B.

FIG. 4 shows the expression levels of three cell surface channels, GLUT-1, MCT-1 and MCT-4 as described in Example 2. Specifically, expression levels of these three cell surface channels were compared in 7 pancreatic cancer cell lines using an RNA expression atlas as shown in FIG. 4A. While neither GLUT1 nor MCT-4 expression levels of CFPAC-1 were lower than other cell lines in the panel, expression levels of MCT-1 in CFPAC-1 (22 TPM) was nearly three-fold lower than the next lowest expressor (BxPC-3, 64 TPM) in the cell line panel as shown in FIG. 4B. FIG. 4C shows that MiaPaCa-2 cells co-treated with varying doses of AZD3965, a known inhibitor of MCT-1, concurrent with CD-3BP showed a clear dose dependent rescue of cells from CD-3BP mediated cytotoxicity.

FIG. 5 shows that the treatment of DLD-1 parental cells with various doses of 3BP (0-200 uM) as described in Example 4 caused dramatic cell death in the parental clones (DLD-1 parental clones), whereas DLD-1 MCT-1(-) (knock-out) cells remained completely resistant to 3BP, even at the 200 uM, with no loss of viability.

FIG. 6 the testing of five commercially available antibodies for specificity as described in Example 5 using DLD-1 parental cells as positive controls and MCT-1 (-) clones. FIG. 6A shows MCT-1 staining in DLD-1 parental clones and FIG. 6B shows MCT-1 staining in DLD-1 MCT-1(-). The scale bar equals to 50 um.

FIG. 7 shows the pattern of MCT-1 expression in several normal tissues as described in Example 5. It was determined that normal testes (FIG. 7A) and gastrointestinal tract, specifically, the colon (FIG. 7B), displayed the highest MCT-1 expression levels whereas normal human pancreas exhibited negligible MCT-1 expression (FIG. 7C). Image taken at 20×.

FIG. 8 shows diffusely high MCT-1 expression in pancreatic adenocarcinoma as described in Example 5.

FIG. 9 shows a line chart representing the viability of MiaPaCa-2 cells at 36 hours after exposure to different doses of sCD-3BP and 3BP for 15 minutes (FIG. 9A), 30 minutes (FIG. 9B), and 60 minutes (FIG. 9C), respectively.

FIG. 10 shows an experimental design from Example 6. Mice were randomized in three arms. The control group received 200 uL of PBS (vehicle). The two treatment arms received 400 and 500 mg/Kg of sCD-3BP in 200 ul PBS. The weights of the animals were assessed the morning of the treatment and drug aliquots in PBS were prepared fresh to administer the appropriate dose depending on the animal's weight.

FIG. 11 shows graphs representing the evolution of bioluminescence in absolute value by average radiance (FIG. 11A) and fold change values of the average radiance over time (FIG. 11B). Treatment started on Day 1.

FIG. 12 shows panels depicting the in vivo bioluminescence of control, 400 mg/Kg sCD-3BP, and 500 mg/kg sCD-3BP during the first week of treatment (upper panels) and 5 days post treatment conclusion (lower panels). Images within panels were taken 13 minutes after injection of 150 uL of D-luciferin, at equal exposure time and aperture (f/stop). Mice are placed following the same order. The tumor in one of the control mice did not show growth and is considered a no take.

FIG. 13 shows pathological results after necropsies. Ex-vivo necropsies were performed to evaluate tumor burden by gross pathology and histology. FIG. 13A shows the gross pathology of tumor burden for each mice in controls and 400 mg/Kg sCD-3BP treatment arm. Several tumor masses were found at the pancreas or peritoneal wall of the control group while only one or fewer number of masses were encountered in the treatment group. FIG. 13B shows the dot plot graph showing differences in total cumulative weights between control and 400 mg/Kg sCD-3BP treatment arms. All the tumor burden was included. FIG. 13C shows each tumor was processed and stained with Hematoxylin and Eosin (H&E) for microscopic histological evaluation. 10 cm ruler included for size comparison. Treated group is smaller in size.

FIG. 14 shows microscopic pictures of histological slides showing characteristics that recapitulate the behavior of Panc02.13 orthotopic tumor. FIG. 14A shows 4× image of a control tumor with pancreatic tissue invasion by tumor cells (asterisk). FIG. 14B shows 10× image of a treated tumor with engulfment of pancreatic islets by cancer cells.

FIG. 15 shows histopathological evaluation of subcutaneous tumors in donor mice. The PDX from Jackson Laboratories were maintained by subcutaneous implantation in NSG mice in our laboratories. FIGS. 15A and 15B show H&E and immunohistochemistry (IHC) for TMO1212 showing an intermediate intensity of MCT-1 immunostaining. FIGS. 15C and 15D show H&E and IHC for TMO1098 showing a high intensity of MCT-1 immunostaining. Note the difference between the tumor capsule (asterisk) and the proper tumor. Images taken at 10×, the scale bars equal to 500 um.

FIG. 16 shows two representative tissues from tissue microarrays (HPanA1500S03 and BC001130, BioMax U.S.) to confirm similar IHC immunostaining in a random set of 100 cases of pancreatic carcinoma as described in Example 5. FIG. 16A shows tissue from a 61-year-old man with PDAC T2N1 M0 showing intermediate intensity of MCT-1 immunostaining. FIG. 16B shows tissue from a 52-year-old man with PDAC T2NOMO showing high intensity of MCT-1 immunostaining.

FIG. 17 shows ultrasound images showing the visualization of the same tumor two weeks apart. The tumor is found deep to the peritoneal wall (pw) and engulfed by the pancreatic tissue (asterisk). Tumor measurements are represented with a dotted line. FIG. 17A shows the early time point. FIG. 17B shows the later time point. Scale bar equals to 15 mm.

FIG. 18 shows experimental design from Example 7Mice were stratified and groups randomized into two arms. The control group received 270 mg/kg sCD in 200 ul PBS (vehicle). The treatment arm received 300 mg/Kg of sCD-3BP in 200 ul PBS. The weights of the animals were assessed the morning of the treatment and drug dilutions in PBS were prepared to administer the appropriate dose depending on the animal's weight.

FIG. 19A provides graphic representation showing tumor growth of TM01212 implanted orthotopically in control mice compared to mice treated with sCD-3BP (300 mg/kg) for 4 weeks. FIG. 19B shows dot plot showing cumulative tumor burden upon necropsy. Tumor weights highlighted in red were probably animals in which TM01212 did not grow properly and highlighted in orange are tumor weights from animals which got 70% lower dose than their cohort counterparts.

FIG. 20 shows gross pathology of excised tumors from TMO1212 mice showing final number and size of lesions. Tumor masses boxed in red were probably animals in which TM01212 did not grow properly and highlighted in yellow are tumor weights from animals, which got 70% lower dose than their cohort counterparts did. Scale bar equals to 1 cm.

FIG. 21A shows dot plot showing cumulative tumor burden upon necropsy excluding no takes and under-dosed animals. FIG. 21 B shows box and whisker plot of tumor weights from control vs treated group. FIG. 21C shows dot plot of macroscopically observable metastatic lesions (number of discrete lesions) in control vs treated group.

FIG. 22 shows histopathological evaluation of orthotopic tumors and metastatic sites in TMO1212 control mice. FIG. 22A shows the H&E of a control tumor at the pancreas highlighting important features such as duct formation (arrow), and distant invasion (asterisk) of normal pancreatic tissue (p). Image taken at 10×, the scale bar equals to 1 mm. FIG. 22B shows IHC with anti-MCT-1 antibody of the same orthotopic tumor depicts the presence of intermediate immunoreactivity as expected. Image taken at 10×, the scale bar equals to 500 um. FIG. 22C shows the H&E of the lung of a control mouse showing many metastases (asterisks). Image taken at 10×, the scale bar equals to 1 mm. FIG. 22D shows the H&E of the liver of a control mouse showing metastases (arrows). Image taken at 20×, the scale bar equals to 200 um.

### DETAILED DESCRIPTION

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “administering” means providing a pharmaceutical agent or composition to a subject, and includes, but is not limited to, administering by a medical professional and self-administering.

The term “3-bromopyruvate” or “3-BP” refers to 3-bromopyruvate, analogs and derivatives of 3-brompyruvate, prodrugs of 3-bromopyruvate, metabolites of 3-bromopyruvate and salts thereof.

The term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

The term “cytotoxic receptor binding small-molecule” refers to any molecule having a molecular weight of less than 1 kDa that binds to MCT-1 and results in cytotoxicity upon entering a cell. Examples of a cytotoxic receptor binding small-molecule include haloacetate, halopyruvate, halolactate, halopropionate or halobutyrate and combinations thereof.

The term “inhibit” or “inhibits” means to decrease, suppress, attenuate, diminish, arrest, or stabilize the development or progression of a disease, disorder, or condition, the activity of a biological pathway, or a biological activity, such as the growth of a solid malignancy, e.g., by at least 10%, 20%, 30%, 40%, 50%, 60%, 70%, 80%, 90%, 95%, 98%, 99%, or even 100% compared to an untreated control subject, cell, biological pathway, or biological activity or compared to the target, such as a growth of a solid malignancy, in a subject before the subject is treated. By the term “decrease” is meant to inhibit, suppress, attenuate, diminish, arrest, or stabilize a symptom of a cancer disease, disorder, or condition. It will be appreciated that, although not precluded, treating a disease, disorder or condition does not require that the disease, disorder, condition or symptoms associated therewith be completely eliminated.

The term “monocarboxylate transporter 1” or “MCT-1” refers to a protein that belongs to the family of monocarboxylate transporter (MCT) transmembrane proteins (which includes not only MCT-1 but also MCT-2, MCT-3 and MCT-4) that mediate the proton-linked bi-directional movement of lactate (as well as pyruvate and ketone bodies) in and out of cells. MCT-1 is encoded by the gene SLC16A1 (1p13.2) and has been well characterized. The expression of MCT-1 has been found throughout nearly all tissues in the human body, with the most notable exception being the endocrine pancreatic beta cells. With regards to cellular localization, MCT-1 has been found to predominantly localize to the plasma membrane as well as the nuclear, sarcolemmal and mitochondrial membranes. In addition, MCT-1 is known to be overexpressed in a number of cancers, including breast, colorectal, pancreatic and cholangiocarcinomas.

The term “modulation” refers to upregulation (i.e., activation or stimulation), downregulation (i.e., inhibition or suppression) of a response, or the two in combination or apart.

The terms “parenteral administration” and “administered parenterally” as used herein mean modes of administration other than enteral and topical administration, usually by injection, and includes, without limitation, intravenous, intrathecal, intramuscular, intraarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The term “pharmaceutically-acceptable salts” refers to the relatively non-toxic, inorganic and organic salts of compounds.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition

A “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “subject suspected of having” means a subject exhibiting one or more clinical indicators of a disease or condition. In certain embodiments, the disease or condition is cancer. In certain aspects, the cancer is a cancer expressing MCT-1. In other aspects, the cancer is leukemia, liver cancer, pancreatic cancer, bile duct cancer, colorectal cancer, mesothelioma, germ cell tumors, glioma, lung cancer, ovarian cancer, prostate cancer, head and neck cancers, melanoma, stomach cancer, bone cancer, renal cancer, bladder cancer, and breast cancer. In some aspects, when the cancer is breast cancer, the cancer is triple negative breast cancer.

The term “subject in need thereof” means a subject identified as in need of a therapy or treatment.

The terms “systemic administration,” “administered systemically,” “peripheral administration,” and “administered peripherally” mean the administration of a compound, drug or other material other than directly into the central nervous system, such that it enters the patient's system and, thus, is subject to metabolism and other like processes, for example, subcutaneous administration.

The term “therapeutic agent” or “pharmaceutical agent” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents is known in the art and may be identified by their effects. Certain therapeutic agents are capable of regulating red cell proliferation and differentiation. Examples include chemotherapeutic nucleotides, drugs, hormones, non-specific (e.g. non-antibody) proteins, oligonucleotides (e.g., antisense oligonucleotides that bind to a target nucleic acid sequence (e.g., mRNA sequence)), peptides, and peptidomimetics.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present disclosure may be administered in a sufficient amount to produce a reasonable benefit/risk ratio applicable to such treatment.

The terms “therapeutically-effective amount” and “effective amount” as used herein means that amount of a compound, material, or composition comprising a compound of the present disclosure which is effective for producing some desired therapeutic effect in at least a sub-population of cells in an animal at a reasonable benefit/risk ratio applicable to any medical treatment.

The term “treating” a disease in a subject or “treating” a subject having a disease refers to subjecting the subject to a pharmaceutical treatment, e.g., the administration of a drug, such that at least one symptom of the disease is decreased or prevented from worsening.

The terms “tumor,” “solid malignancy,” or “neoplasm” refer to a lesion that is formed by an abnormal or unregulated growth of cells. Preferably, the tumor is malignant, such as that formed by a cancer.

### 2. Cyclodextrins

The term “cyclodextrin” refers to a family of cyclic oligosaccharides composed of 5 or more α-D-glucopyranoside units linked together by C1-C4 bonds having a toroidal topological structure, wherein the larger and the smaller openings of the toroid expose certain hydroxyl groups of the α-D-glucopyranoside units to the surrounding environment (e.g., solvent). The term “inert cyclodextrin” refers to a cyclodextrin containing α-D-glucopyranoside units having the basic formula C₆H₁₂O₆ and glucose structure without any additional chemical substitutions (e.g., α-cyclodextrin having 6 glucose monomers, β-cyclodextrin having 7 glucose monomers, and γ-cyclodextrin having 8 glucose monomers). The term “cyclodextrin internal phase” refers to the relatively less hydrophilic region enclosed within (i.e., encapsulated by) the toroid topology of the cyclodextrin structure. The term “cyclodextrin external phase” refers to the region not enclosed by the toroid topology of the cyclodextrin structure and can include, for example, the aqueous environment present during systemic administration in vivo or to the internal phase of a structure that itself encapsulates the cytotoxic receptor binding small-molecule/cyclodextrin complex. Cyclodextrins are useful for solubilizing hydrophobic compositions (see, for example, Albers and Muller (1995) Crit. Rev. Therap. Drug Carrier Syst. 12:311-337; Zhang and Ma (2013) Adv. Drug Delivery Rev. 65:1215-1233; Laza-Knoerr et al. (2010) J. Drug Targ. 18:645-656; Challa et al. (2005) AAPS PharmSci. Tech. 6:E329-357; Uekama et al. (1998) Chem. Rev. 98:2045-2076; Szejtli (1998) Chem. Rev. 98:1743-1754; Stella and He (2008) Toxicol. Pathol. 36:30-42; Rajewski and Stella (1996) J. Pharm. Sci. 85:1142-1169; Thompson (1997) Crit. Rev. Therap. Drug Carrier Sys. 14:1-104; and Irie and Uekama (1997) J. Pharm. Sci. 86:147-162). Any substance located within the cyclodextrin internal phase is said to be “encapsulated.”

As used herein, a cyclodextrin is useful according to the present disclosure so long as the cyclodextrins can encapsulate one or more cytotoxic receptor binding small-molecules. In some embodiments, the cyclodextrin further bears ionizable (e.g., weakly basic and/or weakly acidic) functional groups to enhance the stabilization of the cytotoxic receptor binding small-molecule. By protecting the stability of the cytotoxic receptor binding small-molecule, it is meant that the cytotoxic receptor binding small-molecule/cyclodextrin complex makes the cytotoxic receptor binding small-molecule more stable as seen by photo stability, shelf life stability, thermal stability, stability against intramolecular cyclization, stability to acid hydrolysis, stability against general degradation, and the like, as compared to the stability of a cytotoxic receptor binding small-molecule that is not in a complex with cyclodextrin.

For encapsulating a desired therapeutic agent, cyclodextrins can be selected and/or chemically modified according to the characteristics of the desired therapeutic agent and parameters for efficient, high-concentration loading therein. For example, it is preferable that the cyclodextrin itself have high solubility in water in order to facilitate loading of a therapeutic agent, such as a cytotoxic receptor binding small-molecule. In some embodiments, the water solubility of the cyclodextrin is at least 10 mg/mL, 20 mg/mL, 30 mg/mL, 40 mg/mL, 50 mg/mL, 60 mg/mL, 70 mg/mL, 80 mg/mL, 90 mg/mL, 100 mg/mL or higher. Methods for achieving such enhanced water solubility are well known in the art.

In some embodiments, a large association constant with the therapeutic agent is preferable and can be obtained by selecting the number of glucose units in the cyclodextrin based on the size of the therapeutic agent (see, for example, Albers and Muller (1995) Crit. Rev. Therap. Drug Carrier Syst. 12:311-337; Stella and He (2008) Toxicol. Pathol. 36:30-42; and Rajewski and Stella (1996) J. Pharm. Sci. 85:1142-1169). As a result, the solubility (nominal solubility) of the therapeutic agent in the presence of cyclodextrin can be further improved. For example, the association constant of the cyclodextrin with the therapeutic agent can be 100, 200, 300, 400, 500, 600, 700, 800, 900, 1,000, or higher.

Derivatives formed by reaction with cyclodextrin hydroxyl groups (e.g., those lining the upper and lower ridges of the toroid of an inert cyclodextrin) are readily prepared and offer a means of modifying the physicochemical properties of the parent (inert) cyclodextrin. In some embodiments, the physicochemical properties of the inert cyclodextrin molecule or cyclodextrin molecule that is not complexed with a cytotoxic receptor binding small-molecule differ from the properties of a cyclodextrin molecule complexed with the cytotoxic receptor binding small-molecule. Accordingly, the cytotoxic receptor binding small-molecules complexed with cyclodextrin can be characterized by observing changes in solubility, chemical reactivity, UV/VIS absorbance, drug retention, chemical stability, and the like. For example, it has been determined herein that modifying hydroxyl groups, such as those facing away from the cyclodextrin interior phase, can be replaced with ionizable chemical groups to facilitate loading of therapeutic agents, such as poorly soluble or hydrophobic agents, within the modified cyclodextrins and stabilization thereof. In one embodiment, a modified cyclodextrin having at least one hydroxyl group substituted with an ionizable chemical group will result in a charged moiety under certain solvent (e.g., pH) conditions. The term “charged cyclodextrin” refers to a cyclodextrin having one or more of its hydroxyl groups substituted with a charged moiety and the moiety bearing a charge. Such a moiety can itself be a charged group or it can comprise an organic moiety (e.g., a C₁-C₆ alkyl or C₁-C₆ alkyl ether moiety) substituted with one or more charged moieties.

In one embodiment, the “ionizable” or “charged” moieties are weakly ionizable. Weakly ionizable moieties are those that are either weakly basic or weakly acidic. Weakly basic functional groups (X) have a pKₐ of between about 6.0-9.0, 6.5-8.5, 7.0-8.0, 7.5-8.0, and any range in between inclusive according to CH₃-X. Similarly, weakly acidic functional groups (Y) have a log dissociation constant (pKₐ) of between about 3.0-7.0, 4.0-6.5, 4.5-6.5, 5.0-6.0, 5.0-5.5, and any range in between inclusive according to CH₃-Y. The pKₐ parameter is a well-known measurement of acid/base properties of a substance and methods for pKₐ determination are conventional and routine in the art. For example, the pKₐ values for many weak acids are tabulated in reference books of chemistry and pharmacology. See, for example, IUPAC Handbook of Pharmaceutical Salts, ed. by P. H. Stahl and C. G Wermuth, Wiley-VCH, 2002; CRC Handbook of Chemistry and Physics, 82nd Edition, ed. by D. R. Lide, CRC Press, Florida, 2001, p. 8-44 to 8-56. Since cyclodextrins with more than one ionizable group have pKₐ of the second and subsequent groups each denoted with a subscript.

Representative anionic moieties include, without any limitation, succinyl, carboxylate, carboxymethyl, sulfonyl, phosphate, sulfoalkyl ether, sulphate carbonate, thiocarbonate, thiocarbonate, phosphate, phosphonate, sulfonate, nitrate, and borate groups.

Representative cationic moieties include, without limitation, amino, guanidine, and quaternary ammonium groups.

In another embodiment, the modified cyclodextrin is a “polyanion” or “polycation.” A polyanion is a modified cyclodextrin having more than one negatively charged group resulting in net negative ionic charger of more than two units. A polycation is a modified cyclodextrin having more than one positively charged group resulting in net positive ionic charger of more than two units.

In another embodiment, the modified cyclodextrin is a “chargeable amphiphile.” By “chargeable” is meant that the amphiphile has a pK in the range pH 4 to pH 8 or 8.5. A chargeable amphiphile may therefore be a weak acid or base. By “amphoteric” herein is meant a modified cyclodextrin having a ionizable groups of both anionic and cationic character wherein: 1) at least one, and optionally both, of the cation and anionic amphiphiles is chargeable, having at least one charged group with a pK between 4 and 8 to 8.5, 2) the cationic charge prevails at pH 4, and 3) the anionic charge prevails at pH 8 to 8.5.

In some embodiments, the “ionizable” or “charged” cyclodextrins as a whole, whether polyionic, amphiphilic, or otherwise, are weakly ionizable (i.e., have a pKₐ₁ of between about 4.0-8.5, 4.5-8.0, 5.0-7.5, 5.5-7.0, 6.0-6.5, and any range in between inclusive).

Any one, some, or all hydroxyl groups of any one, some or all α-D-glucopyranoside units of a cyclodextrin can be modified to an ionizable chemical group as described herein. Since each cyclodextrin hydroxyl group differs in chemical reactivity, reaction with a modifying moiety can produce an amorphous mixture of positional and optical isomers. Alternatively, certain chemistry can allow for pre-modified α-D-glucopyranoside units to be reacted to form uniform products.

The aggregate substitution that occurs is described by a term called the degree of substitution. For example, a 6-ethylenediamino-β-cyclodextrin with a degree of substitution of seven would be composed of a distribution of isomers of 6-ethylenediamino-β-cyclodextrin in which the average number of ethylenediamino groups per 6-ethylenediamino-β-cyclodextrin molecule is seven. Degree of substitution can be determined by mass spectrometry or nuclear magnetic resonance spectroscopy. Theoretically, the maximum degree of substitution is 18 for α-cyclodextrin, 21 for 3, and 24 for γ-cyclodextrin, however, substituents themselves having hydroxyl groups present the possibility for additional hydroxylalkylations. The degree of substitution can be 1, 2, 3, 4, 5, 6, 7, 8, 9, 10, 11, 12, 13, 14, 15, 16, 17, 18, 19, 20, 21, 22, 23, 24, or more and can encompass complete substitution.

Another parameter is the stereochemical location of a given hydroxyl substitution. In one embodiment, at least one hydroxyl facing away from the cyclodextrin interior is substituted with an ionizable chemical group. For example, the C2, C3, C6, C2 and C3, C2 and C6, C3 and C6, and all three of C2-C3-C6 hydroxyls of at least one α-D-glucopyranoside unit are substituted with an ionizable chemical group. Such carbon positions are well known in the art. For example, the CH2OH moiety of each α-D-glucopyranoside unit represents the C6 carbon. Any such combination of hydroxyls can similarly be combined with at least two, three, four, five, six, seven, eight, nine, ten, eleven, up to all of the α-D-glucopyranoside units in the modified cyclodextrin as well as in combination with any degree of substitution described herein.

### 3. Cytotoxic Receptor Binding Small-Molecules

The term “cytotoxic receptor-binding small molecule” as used herein refers to any molecule having a molecular weight of less than 1 kDa that binds to MCT-1 and results in cytotoxicity upon entering a cell. Methods for identifying whether molecules having a molecular weight of less than 1 kDa and that bind to MCT-1 result in cytotoxicity upon entering a cell are well known in the art. For example, an ALT-R CRISPR system can be used to knock out SLC16A1, the gene responsible for encoding the receptor protein MCT-1 in cell lines that express high levels of it, using routine techniques known in the art, producing corresponding clones that do not express any MCT-1 protein and therefore, the receptor knocked out and the clone devoid of MCT-1 function. Using these paired isogenic cell lines, a drug screening can be performed against a library of small molecules. More specifically, each parental/MCT-1 knock-out (KO) pair can be exposed to same drug at an identical concentration and the viability of the lines assessed. All agents that selectively reduce viability of parental cell line while preserving the corresponding MCT-1 KO clone will then be considered a cytotoxic receptor binding small molecule. Each agent can then be evaluated further at multiple doses to assess the therapeutic window in vitro prior to testing in vivo in established models for efficacy and toxicity.

Examples of a cytotoxic receptor binding small-molecule include haloacetate, halopyruvate, halolactate, halopropionate or halobutyrate and combinations thereof.

### 4. Cyclodextrin/Cytotoxic Receptor Binding Small-Molecule Compositions

The present disclosure provides pharmaceutical compositions comprising at least one cytotoxic receptor binding small-molecule described above encapsulated within inert and/or modified cyclodextrins. Such complexes are referred to herein as cyclodextrin/cytotoxic receptor binding small-molecule compositions. The ratio of cytotoxic receptor binding small-molecule to cyclodextrin may be 1:1 such that one inhibitor molecule forms a complex with one cyclodextrin molecule. Alternatively, the ratio can be 2:1, 3:1, 4:1, 5:1, or more.

In one aspect, the present disclosure provides pharmaceutically acceptable compositions which comprise a therapeutically-effective amount of one or more such cyclodextrin/cytotoxic receptor binding small-molecules described above, formulated together with one or more pharmaceutically acceptable carriers (additives) and/or diluents. In another aspect the compositions can be administered as such or in admixtures with pharmaceutically acceptable carriers and can also be administered in conjunction with other anti-cancer therapies, such as chemotherapeutic agents, scavenger compounds, radiation therapy, biologic therapy, and the like. Conjunctive therapy thus includes sequential, simultaneous and separate, or co-administration of the composition, wherein the therapeutic effects of the first administered has not entirely disappeared when the subsequent compound is administered.

As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous, intrathecal, or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

As set out above, certain embodiments of the at least one cytotoxic receptor binding small-molecules or cyclodextrin/ cytotoxic receptor binding small-molecule compositions may contain a basic functional group, such as amino or alkylamino, and are, thus, capable of forming pharmaceutically-acceptable salts with pharmaceutically-acceptable acids. These salts can be prepared in situ in the administration vehicle or the dosage form manufacturing process, or by separately reacting a purified compound of the disclosure in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the cytotoxic receptor binding small-molecules or cyclodextrin/cytotoxic receptor binding small-molecule compositions of the present disclosure may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrin/cytotoxic receptor binding small-molecule composition formulations include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, a formulation of cyclodextrin/cytotoxic receptor binding small-molecule compositions can comprise other carriers to allow more stability, to allow more stability, different releasing properties in vivo, targeting to a specific site, or any other desired characteristic that will allow more effective delivery of the complex to a subject or a target in a subject, such as, without limitation, liposomes, microspheres, nanospheres, nanoparticles, bubbles, micelle forming agents, e.g., bile acids, and polymeric carriers, e.g., polyesters and polyanhydrides. In certain embodiments, an aforementioned formulation renders orally bioavailable a compound of the present disclosure.

Liquid dosage formulations of cyclodextrin/cytotoxic receptor binding small-molecule compositions include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an active ingredient. A cyclodextrin/cytotoxic receptor binding small-molecule composition of the present disclosure may also be administered as a bolus, electuary or paste.

In solid dosage forms (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate.

Dosage forms for the topical or transdermal administration of a cyclodextrin/cytotoxic receptor binding small-molecule composition of the present disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure.

Pharmaceutical compositions suitable for parenteral administration can comprise sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In certain embodiments, the above-described pharmaceutical compositions can be combined with other pharmacologically active compounds (“second active agents”) known in the art according to the methods and compositions provided herein. Second active agents can be large molecules (e.g., proteins) or small molecules (e.g., synthetic inorganic, organometallic, or organic molecules). In one embodiment, second active agents independently or synergistically help to treat cancer.

For example, chemotherapeutic agents are anti-cancer agents. The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

Further, the following drugs may also be used in combination with an antineoplastic agent, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used: interleukins 1 through 18, including mutants and analogues; interferons or cytokines, such as interferons α, β, and γ; hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In another embodiment, the composition of the disclosure may comprise other biologically active substances, including therapeutic drugs or pro-drugs, for example, other chemotherapeutic agents, scavenger compounds, antibiotics, anti-virals, anti-fungals, anti-inflammatories, vasoconstrictors and anticoagulants, antigens useful for cancer vaccine applications or corresponding pro-drugs.

Exemplary scavenger compounds include, but are not limited to thiol-containing compounds such as glutathione, thiourea, and cysteine; alcohols such as mannitol, substituted phenols; quinones, substituted phenols, aryl amines and nitro compounds.

Various forms of the chemotherapeutic agents and/or other biologically active agents may be used. These include, without limitation, such forms as uncharged molecules, molecular complexes, salts, ethers, esters, amides, and the like, which are biologically active.

### 5. Methods of Making Cyclodextrin/Cytotoxic Receptor Binding Small-Molecule Compositions

Methods of preparing cyclodextrin/cytotoxic receptor binding small-molecule compositions and formulations thereof include the step of bringing into association a compound of the present disclosure with the carrier and, optionally, one or more accessory ingredients. In general, the formulations are prepared by uniformly and intimately bringing into association a cytotoxic receptor binding small-molecule described herein with a cyclodextrin. Generally, such complexes can be obtained by agitating and mixing the cyclodextrin (e.g., a solution containing the cyclodextrin) upon dropwise addition of the therapeutic agent (e.g., a solution containing one or more cytotoxic receptor binding small-molecules) or vice versa. Many mixing means are known in the art to aid in combining the inhibitor and cyclodextrin for example, without limitation, sonication, vortexing, stirring, heating, co-precipitation, neutralization, slurrying, kneading, grinding, and the like. It is possible to use a substance dissolved in a solvent or a solid substance as the therapeutic agent according to the physical properties of the therapeutic agent. There are no particular limitations on the solvent, and one can use, for example, a substance identical to the cyclodextrin external phase. The amount of the therapeutic agent that is mixed with the cyclodextrin can be equimolar quantities or in different ratios depending on the desired level of incorporation. In some embodiments, absolute amounts of the cytotoxic receptor binding small molecule can range between 0.01 to 1 mol equivalent, or any range inclusive relative to the amount of cyclodextrin Also, the only particular limitations on the heating temperature is that the heating not occur higher than room temperature.

Well-known methods exist for removing any undesired or unincorporated complexes or compositions, such as therapeutic agent not encapsulated by cyclodextrins or therapeutic agent cyclodextrin complexes not encapsulated by liposomes. Representative examples include, without limitation, dialysis, centrifugal separation, and gel filtration. Dialysis can be conducted, for example, using a dialysis membrane. As a dialysis membrane, one may cite a membrane with molecular weight cut-off such as a cellulose tube or Spectra/Por. With respect to centrifugal separation, centrifugal acceleration any be conducted preferably at 100,000 g or higher, and more preferably at 300,000 g or higher. Gel filtration may be carried out, for example, by conducting fractionation based on molecular weight using a column such as Sephadex or Sepharose.

In some cases, in order to prolong the effect of a drug, it is desirable to modify (e.g., slow) the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form can be accomplished by dissolving or suspending the drug in an oil vehicle. In some embodiments, the cyclodextrin-encapsulated cytotoxic receptor binding small-molecule compositions described herein can be loaded into liposomes.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

### 6. Therapeutic Methods

The present disclosure further provides novel therapeutic methods of preventing, delaying, reducing, and/or treating a cancer, including a cancerous tumor. In one embodiment, a method of treatment comprises administering to a subject (e.g., a subject in need thereof), an effective amount of a cyclodextrin/cytotoxic receptor binding small-molecule composition. A subject in need thereof may include, for example, a subject who has been diagnosed with a tumor, including a pre-cancerous tumor, a cancer, or a subject who has been treated, including subjects that have been refractory to the previous treatment.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

The methods of the present disclosure may be used to treat any cancerous or pre-cancerous tumor. More specifically, the methods of the present disclosure can be used to treat any cancerous or pre-cancerous tumor expressing MCT-1. For example, cancerous or pre-cancerous tumors that can be treated include liver cancer, pancreatic cancer, bile duct cancer, colorectal cancer, mesothelioma, leukemias (e.g, such as ; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia), germ cells tumors, glioma, medulloblastoma, neuroblastoma, lung cancer, ovarian cancer, prostate cancer, head and neck cancers, melanoma, stomach cancer, bone cancer, renal cancer, bladder cancer, and breast cancer. In some aspects, when the cancer is breast cancer it is triple negative breast cancer.

The compositions described herein may be delivered by any suitable route of administration, including orally, nasally, transmucosally, ocularly, rectally, intravaginally, parenterally, including intramuscular, subcutaneous, intramedullary injections, as well as intrathecal, direct intraventricular, intravenous, intrathecal, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

In certain embodiments the pharmaceutical compositions are delivered generally (e.g., via oral or parenteral administration). In certain other embodiments the pharmaceutical compositions are delivered locally through direct injection into a tumor or direct injection into the tumor's blood supply (e.g., arterial or venous blood supply). In some embodiments, the pharmaceutical compositions are delivered by both a general and a local administration. For example, a subject with a tumor may be treated through direct injection of a composition containing a composition described herein into the tumor or the tumor's blood supply in combination with oral administration of a pharmaceutical composition of the present disclosure. If both local and general administration is used, local administration can occur before, concurrently with and/or after general administration.

In certain embodiments, the methods of treatment of the present disclosure, including treating a cancerous or pre-cancerous tumor comprise administering compositions described herein in combination with a second agent and/or therapy to the subject. By “in combination with” is meant the administration of the cytotoxic receptor binding small-molecule/cyclodextrin complexes with one or more therapeutic agents either simultaneously, sequentially, or a combination thereof. Therefore, a subject administered a combination of the cytotoxic receptor binding small-molecules/cyclodextrin complexes and/or therapeutic agents, can receive the cytotoxic receptor binding small-molecule/cyclodextrin complexes as described herein, and one or more therapeutic agents at the same time (i.e., simultaneously) or at different times (i.e., sequentially, in either order, on the same day or on different days), so long as the effect of the combination of both agents is achieved in the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

Such methods in certain embodiments comprise administering pharmaceutical compositions comprising compositions described herein in conjunction with one or more chemotherapeutic agents and/or scavenger compounds, including chemotherapeutic agents described herein, as well as other agents known in the art. Conjunctive therapy includes sequential, simultaneous and separate, or co-administration of the composition in a way that the therapeutic effects of the first selective cytotoxic receptor binding small-molecule administered have not entirely disappeared when the subsequent compound is administered. In one embodiment, the second agent is a chemotherapeutic agent. In another embodiment, the second agent is a scavenger compound. In another embodiment, the second agent is radiation therapy. In a further embodiment, radiation therapy may be administered in addition to the composition. In certain embodiments, the second agent may be co-formulated in the separate pharmaceutical composition.

In some embodiments, the subject pharmaceutical compositions of the present disclosure will incorporate the substance or substances to be delivered in an amount sufficient to deliver to a patient a therapeutically effective amount of an incorporated therapeutic agent or other material as part of a prophylactic or therapeutic treatment. The desired concentration of the active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Dosage may be based on the amount of the composition or active compound thereof (e.g., cytotoxic receptor binding small molecules) per kg body weight of the patient. For example, a range of amounts of compositions or compound encapsulated therein are contemplated, including about 0.001, 0.01, 0.1, 0.5, 1, 10, 15, 20, 25, 50, 75, 100, 150, 200 or 250 mg or more of such compositions per kg body weight of the patient. Other amounts will be known to those of skill in the art and readily determined. In certain embodiments, the dosage of the composition or active compound thereof (e.g., cytotoxic receptor binding small molecules) will generally be in the range of about 0.001 mg to about 800 mg per kg body weight, specifically in the range of about 50 mg to about 800 mg per kg, and more specifically in the range of about 100 mg to about 800 mg per kg. In another embodiment, the dosage range is about 50 mg to about 700 mg per kg. In yet another embodiment, the dosage range is about 100 mg to about 700 mg per kg. In yet another embodiment, the dosage range is about 50 mg to about 600 mg per kg. In yet another embodiment, the dosage range is about 100 mg to about 600 mg per kg. In yet another embodiment, the dosage range is about 50 mg to about 500 mg per kg. In yet another embodiment, the dosage range is about 100 mg to about 500 mg per kg. In yet another embodiment, the dosage range is about 50 mg to about 400 mg per kg. In yet another embodiment, the dosage range is about 100 mg to about 400 mg per kg. In yet another embodiment, the dosage range is about 50 mg to about 300 mg per kg. In yet another embodiment, the dosage range is about 100 mg to about 300 mg per kg. In yet another embodiment, the dosage range is about 50 mg to about 200 mg per kg. In yet another embodiment, the dosage range is about 100 mg to about 200 mg per kg. In yet another embodiment, the dosage range is about 50 mg to about 100 mg per kg. In yet another embodiment, the dosage range is about 0.001 mg to about 200 mg per kg. In yet another embodiment, the dosage range is about 0.001 mg to about 100 mg per kg. In yet another embodiment, the dosage range is about 0.1 mg to about 200 mg per kg. In yet another embodiment, the dosage range is about 0.1 mg to about 100 mg per kg. In yet another embodiment, the dosage range is about 1 mg to about 200 mg per kg. In still yet another embodiment, the dosage range is about 1 mg to about 100 mg per kg.

In some embodiments the molar concentration of the composition or active compound thereof (e.g., cytotoxic receptor binding small molecules) in a pharmaceutical composition will be less than or equal to about 2.5 M, 2.4 M, 2.3 M, 2.2 M, 2.1 M, 2 M, 1.9 M, 1.8 M, 1.7 M, 1.6 M, 1.5 M, 1.4 M, 1.3 M, 1.2 M, 1.1 M, 1 M, 0.9 M, 0.8 M, 0.7 M, 0.6 M, 0.5 M, 0.4 M, 0.3 M or 0.2 M. In some embodiments the concentration of the composition or active compound thereof (e.g., cytotoxic receptor binding small molecules) will be less than or equal to about 0.10 mg/ml, 0.09 mg/ml, 0.08 mg/ml, 0.07 mg/ml, 0.06 mg/ml, 0.05 mg/ml, 0.04 mg/ml, 0.03 mg/ml or 0.02 mg/ml.

Alternatively, the dosage may be determined by reference to the plasma concentrations of the composition or active compound thereof (e.g., cytotoxic receptor binding small-molecules). For example, the maximum plasma concentration (Cₘₐₓ) and the area under the plasma concentration-time curve from time 0 to infinity (AUC (0-4)) may be used. Dosages for the present disclosure include those that produce the above values for Cₘₐₓ and AUC (0-4) and other dosages resulting in larger or smaller values for those parameters.

Actual dosage levels of the active ingredients in the compositions of the present disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular therapeutic agent in the formulation employed, or the ester, salt or amide thereof, the route of administration, the time of administration, the rate of excretion or metabolism of the particular therapeutic agent being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds of the disclosure employed in the pharmaceutical composition at levels lower than that required in order to achieve the desired therapeutic effect and gradually increase the dosage until the desired effect is achieved.

In general, a suitable daily dose of a compound of the disclosure will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. All aspects of the treatment, including supplements, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments, for example, to the amount(s) of agent administered and to the time of administration may be made based on these reevaluations.

Treatment may be initiated with smaller dosages which are less than the optimum dose of the compound. Thereafter, the dosage may be increased by small increments until the optimum therapeutic effect is attained.

As described above, the composition or active compound thereof (e.g., cytotoxic receptor binding small-molecules) may be administered in combination with radiation therapy. An optimized dose of radiation therapy may be given to a subject as a daily dose. Optimized daily doses of radiation therapy may be, for example, from about 0.25 to 0.5 Gy, about 0.5 to 1.0 Gy, about 1.0 to 1.5 Gy, about 1.5 to 2.0 Gy, about 2.0 to 2.5 Gy, and about 2.5 to 3.0 Gy. An exemplary daily dose may be, for example, from about 2.0 to 3.0 Gy. A higher dose of radiation may be administered, for example, if a tumor is resistant to lower doses of radiation. High doses of radiation may reach, for example, 4 Gy. Further, the total dose of radiation administered over the course of treatment may, for example, range from about 50 to 200 Gy. In an exemplary embodiment, the total dose of radiation administered over the course of treatment ranges, for example, from about 50 to 80 Gy. In certain embodiments, a dose of radiation may be given over a time interval of, for example, 1, 2, 3, 4, or 5 mins, wherein the amount of time is dependent on the dose rate of the radiation source.

In certain embodiments, a daily dose of optimized radiation may be administered, for example, 4 or 5 days a week, for approximately 4 to 8 weeks. In an alternate embodiment, a daily dose of optimized radiation may be administered daily seven days a week, for approximately 4 to 8 weeks. In certain embodiments, a daily dose of radiation may be given a single dose. Alternately, a daily dose of radiation may be given as a plurality of doses. In a further embodiment, the optimized dose of radiation may be a higher dose of radiation than can be tolerated by the patient on a daily base. As such, high doses of radiation may be administered to a patient, but in a less frequent dosing regimen.

The types of radiation that may be used in cancer treatment are well known in the art and include electron beams, high-energy photons from a linear accelerator or from radioactive sources such as cobalt or cesium, protons, and neutrons. An exemplary ionizing radiation is an x-ray radiation.

Methods of administering radiation are well known in the art. Exemplary methods include, but are not limited to, external beam radiation, internal beam radiation, and radiopharmaceuticals. In external beam radiation, a linear accelerator is used to deliver high-energy x-rays to the area of the body affected by cancer. Since the source of radiation originates outside of the body, external beam radiation can be used to treat large areas of the body with a uniform dose of radiation. Internal radiation therapy, also known as brachytherapy, involves delivery of a high dose of radiation to a specific site in the body. The two main types of internal radiation therapy include interstitial radiation, wherein a source of radiation is placed in the effected tissue, and intracavity radiation, wherein the source of radiation is placed in an internal body cavity a short distance from the affected area. Radioactive material may also be delivered to tumor cells by attachment to tumor-specific antibodies. The radioactive material used in internal radiation therapy is typically contained in a small capsule, pellet, wire, tube, or implant. In contrast, radiopharmaceuticals are unsealed sources of radiation that may be given orally, intravenously or directly into a body cavity.

Radiation therapy may also include stereotactic surgery or stereotactic radiation therapy, wherein a precise amount of radiation can be delivered to a small tumor area using a linear accelerator or gamma knife and three dimensional conformal radiation therapy (3DCRT), which is a computer assisted therapy to map the location of the tumor prior to radiation treatment.

Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the cyclodextrin-encapsulated selective cytotoxic receptor binding small-molecule compositions described herein relative to the selective cytotoxic receptor binding small-molecule without any cyclodextrin encapsulation. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the cyclodextrin-encapsulated selective cytotoxic receptor binding small-molecule compositions described herein relative to the selective cytotoxic receptor binding small-molecule without any cyclodextrin encapsulation. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the cyclodextrin-encapsulated selective cytotoxic receptor binding small-molecule compositions described herein relative to the cytotoxic receptor binding small-molecule without any cyclodextrin encapsulation. Although compounds that exhibit toxic side effects may be used, care should be taken to design a delivery system that targets the compounds to the desired site in order to reduce side effects.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% inhibition of cancer cell growth in an assay.

In any of the above-described methods, the administering of the cytotoxic receptor binding small-molecule/cyclodextrin complexes can result in at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy in a subject, compared to the solid malignancy before administration of the cytotoxic receptor binding small-molecule /cyclodextrin complexes.

In some embodiments, the therapeutically effective amount of a complex of a cytotoxic receptor binding small-molecule/cyclodextrin is administered prophylactically to prevent a solid malignancy from forming in the subject.

In some embodiments, the subject is human. In other embodiments, the subject is non-human, such as a mammal.

The data obtained from the cell culture assays and animal studies may be used in formulating a range of dosage for use in humans. The dosage of any supplement, or alternatively of any components therein, lies preferably within a range of circulating concentrations that include the ED₅₀ with little or no toxicity. The dosage may vary within this range depending upon the dosage form employed and the route of administration utilized. For agents of the present disclosure, the therapeutically effective dose may be estimated initially from cell culture assays. A dose may be formulated in animal models to achieve a circulating plasma concentration range that includes the IC₅₀ as determined in cell culture. Such information may be used to more accurately determine useful doses in humans. Levels in plasma may be measured, for example, by high performance liquid chromatography.

### 7. Kits

The cytotoxic receptor binding small-molecule/cyclodextrin complexes and compositions described herein can be assembled into kits or pharmaceutical systems for use in treating or preventing a disease, such as cancer. In some embodiments, the cytotoxic receptor binding small-molecule-cyclodextrin complex and compositions can be used to prevent or treat solid malignancies caused by a cancer. In general, a presently disclosed kit contains some or all of the components, reagents, supplies, and the like to practice a method according to the presently disclosed subject matter. The kit typically comprises an effective amount of complex to prevent, delay, reduce, or treat an unwanted disease (e.g., a solid malignancy). In one embodiment, a kit comprises at least one container (e.g., a carton, bottle, vial, tube, or ampoule) comprising a cytotoxic receptor binding small-molecule/cyclodextrin complex and/or compositions thereof described herein. Typically, the complex and/or compositions will be supplied in one or more container, each container containing an effective amount of complex to allow a solid malignancy to regress, slow, or be arrested.

Accordingly, in some embodiments, the presently disclosed subject matter provides a kit comprising at least one cytotoxic receptor binding small-molecule encapsulated within at least one cyclodextrin carrier. In other embodiments, the kit further comprises a set of instructions for using the at least one cytotoxic receptor binding small-molecule encapsulated within the at least one cyclodextrin carrier.

It may be desirable to store the cytotoxic receptor binding small-molecule and cyclodextrin separately and then combine them before use. Accordingly, in still other embodiments, the kit comprises at least one cytotoxic receptor binding small-molecule in one container and at least one cyclodextrin carrier in another container.

### 8. Assay

In another embodiment, the present disclosure provides a method for assessing the stability of a composition comprising at least one agent encapsulated in a cyclodextrin, such as a β-cyclodextrin. The method at least the following steps:
providing at least one cyclodextrin (e.g., such as a β-cyclodextrin) and at least one agent, wherein the at least one agent is encapsulated in the at least one cyclodextrin to provide at least one cyclodextrin encapsulated agent composition;
assessing the composition for presence or absence of 3-bromopyruvate using a cell toxicity assay; and
determining the stability of the composition.

Any agent that can be encapsulated with the at least one cyclodextrin can be use in the above method. For example, the at least one agent can be at least one inhibitor of MCT-1 or at least one cytotoxic receptor binding small molecule.

Any cell toxicity assay known in the art can be used in the above method. Examples of a cell toxicity assay that can be used in the above method include the CellTox™ Green Cytotoxicity Assay which is available from Promega (Madison, WI) or the ToxiLight™ Non-Destructive Cytotoxicity Bioassay Kit available from Lonza (Basel, CH).

Additionally, the above method can comprise incubating the composition in sera for at least 30 minutes prior to performing the cell toxicity assay.

### 9. Examples

The present disclosure has multiple aspects, illustrated by the following non-limiting examples.

It is understood that the foregoing detailed description and accompanying examples are merely illustrative and are not to be taken as limitations upon the scope, which is defined solely by the appended claims and their equivalents.

### Example 1: Generation of Panels of CD-3BP Complexes and Stability Evaluation

In order to clarify how different substituents on β-CDs may affect encapsulation efficiency and release rates of 3-bromopyruvate (3BP) from the β-cyclodextrin (β-CD) cavity and subsequently alter biological outcome, CD-3BP complexes were synthesized with two additional cyclodextrins, i) 2-hydroxypropyl-β-CD (HPCD) and ii) sulfobutylether-β-CD (SBECD) (Table 1). Further, CD-3BP was synthesized at different ratios (1:1, 2:1, 3:1, 4:1) of CD: drug to identify an optimized formulation suitable for clinical development. The encapsulation of 3-BP in β-CD was accomplished by portion-wise addition of correct molar equivalent 3-BP to a stirring solution of desired β-CD.

### Method for CD-3BP-1, CD-3BP-2, and CD-3BP-3 (1:1 Complexes)

A solid sample of 3BP (1 mmol, 167 mg) was added in small portions to a solution of the corresponding cyclodextrin (1 mmol) in 25 ml of deionized (DI) water under constant agitation. After complete addition (15-20 minutes), the samples were further agitated for 1-4 hours at 25° C. Finally, the sample was flash frozen using liquid nitrogen or dry ice acetone and lyophilized overnight to produce a 1:1 complex.

### Method for CD-3BP-4, CD-3BP-5, CD-3BP-6

A solid sample of 3BP (1 mmol, 167 mg) was added in small portions to a solution of the corresponding cyclodextrin (2, 3, or 4 mmol) in DI water (40-50 mM concentration) under constant agitation. After complete addition, the samples were further agitated for 1-4 hours at 25° C. Finally, the sample was flash frozen using liquid nitrogen or dry ice acetone and lyophilized overnight to produce the desired complex.

TABLE 1

| No. | CD | CD:3BP |
|---|---|---|
| CD-3BP-1 | succinyl β-CD | 1:1 |
| CD-3BP-2 | sulfobutyl ether β-CD | 1:1 |
| CD-3BP-3 | 2-hydroxypropy-β-CD | 1:1 |
| CD-3BP-4 | succinyl β-CD | 2:1 |
| CD-3BP-5 | succinyl β-CD | 3:1 |
| CD-3BP-6 | succinyl β-CD | 4:1 |
| CD-3BP-7 | sulfobutyl ether β-CD | 2:1 |
| CD-3BP-8 | sulfobutyl ether β-CD | 3:1 |
| CD-3BP-9 | sulfobutyl ether β-CD | 4:1 |

### Size Exclusion HPLC Method to Analyze Complexes

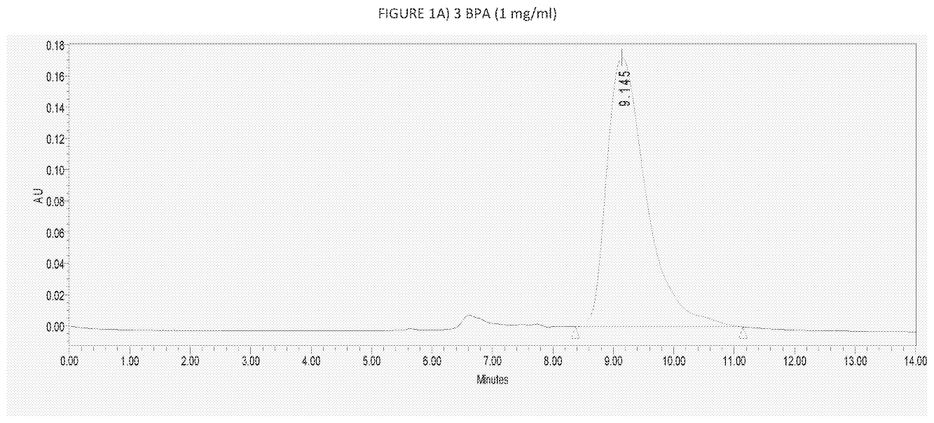

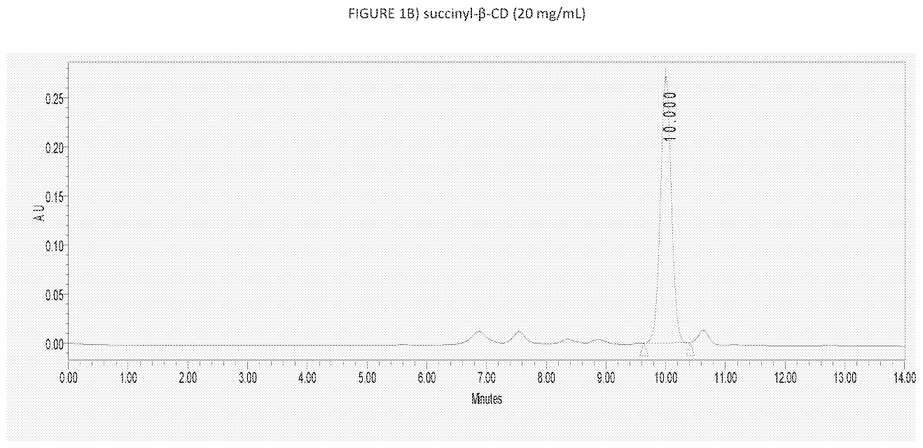

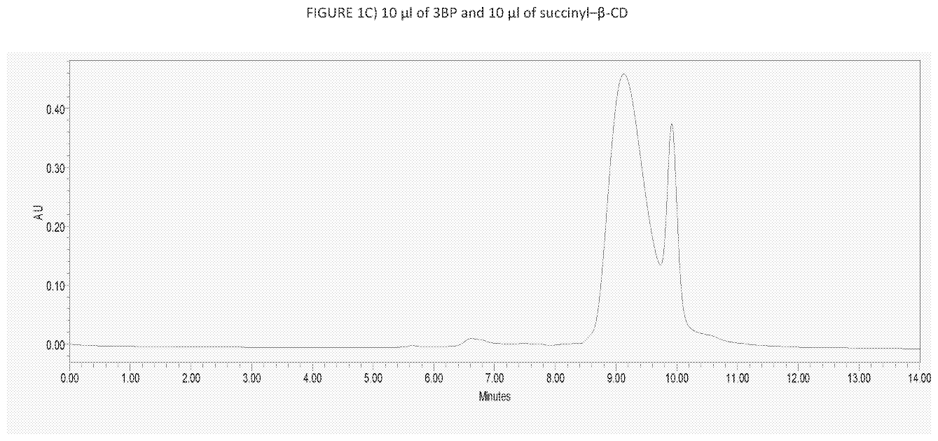

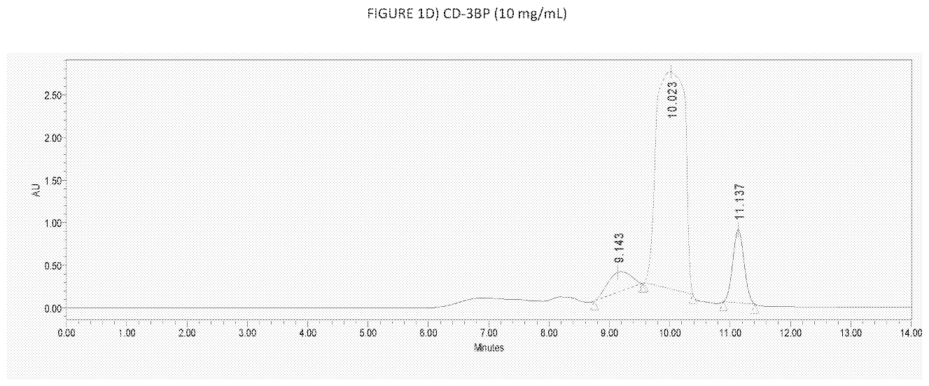

To characterize the microencapsulated complexes, and estimate the level of encapsulation, SEC chromatography using a Shodex-OH Pak column was used. It was hypothesized that upon efficient encapsulation, CD-3BP would show an identical retention time as the parent cyclodextrin used. To test, the individual components were characterized using a Shodex-OH, running at 1 ml/min PBS under isocratic conditions for 15 minutes. Samples used: i) free 3BP (1 mg/mL); ii) succinyl-β-CD (20 mg/mL); iii) a mixture of 10 μL of free 3BP and 10 μL succinyl-β-CD; and iv) CD-3BP (10 mg/mL). Samples i) and ii) were further diluted to 1 mg/mL and 10 mg/mL prior to a 20 μL injection. Samples were monitored at 220 nm as shown in FIGS. 1A-1D. Specifically, free 3BP (1 mg/mL) is shown in FIG. 1A, succinyl-β-CD (20 mg/mL) is shown in FIG. 1B, a mixture of 10 μl of 3BP and 10 μl of succinyl-β-CD is shown in FIG. 1C, and CD-3BP (10 mg/mL) in FIG. 1D.

### Succinyl-β-CD-3BP Protects 3BP from Rapid Degradation by Serum Proteins

A prior study in Chapiro, J. et al., Clinical cancer research: An official journal of the American Association for Cancer Research 20, 6406-6417, doi:10.1158/1078-0432.ccr-14-1271 (2014), has previously demonstrated potent in vivo antitumor activity of CD-3BP with dramatic reduction of toxicity. However, how microencapsulation in CD alters the solubility and stability of 3BP and whether chemical modifications of CDs can alter physicochemical characteristics of the drug remains unclear. To address these questions and clarify the biological effect of microencapsulation, an assay was designed to estimate the stability of various CD-3BP complexes when compared to free 3BP in human sera.

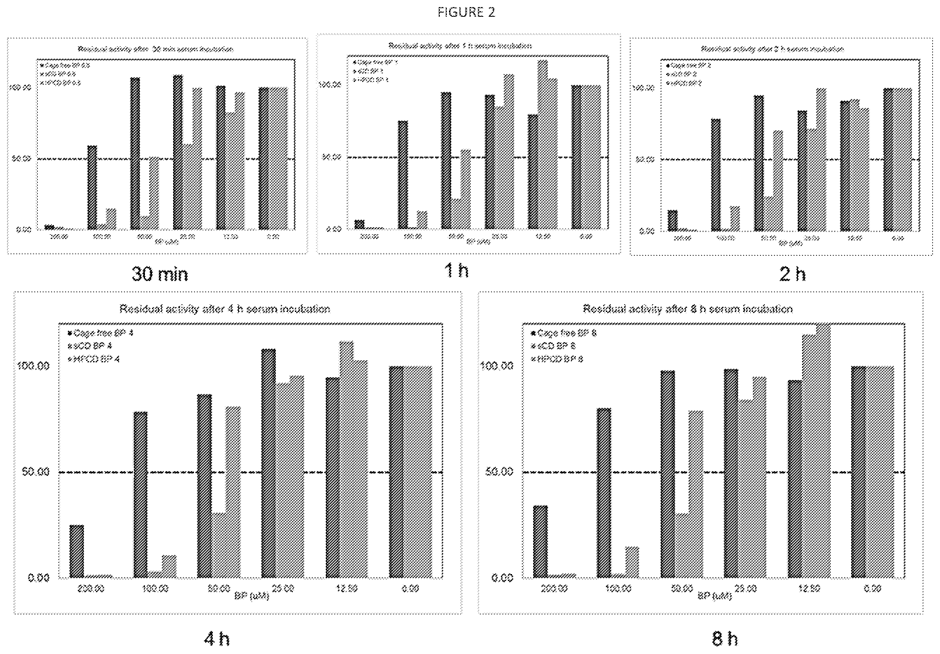

Two different types of complexes, the previously used sCD-3BP (Chapiro, J. et al., Clinical cancer research: An official journal of the American Association for Cancer Research 20, 6406-6417, doi:10.1158/1078-0432.ccr-14-1271 (2014)) and newly generated HPCD-3BP were compared side-by-side. While the internal cavities of both cyclodextrins, (HPCD and sCD) are identical, the surface of these cyclodextrins are markedly different with HPCD bearing neutral hydroxyl propyl groups while sCD carries anionic succinyl moieties. Identical molar amounts of the 3 different agents (free 3BP, sCD-3BP and HPCD-3BP) were incubated with equal amounts of human sera at 37° C. Aliquots were collected at various time points and assessed for residual 3BP by a cell toxicity assay. Free 3BP was rapidly degraded in sera while microencapsulated versions offered different degrees of protection (FIG. 2). The sCD-3BP complex appeared to be the most stable under these conditions for up to 8 hours and maintained near complete levels of activity at various concentrations while HPCD-3BP complex offered intermediate level of protection with significant loss of activity seen after 2 hours (FIG. 2). Given these preliminary results subsequent in vitro and in vivo experiments were conducted using sCD-3BP.

### Method to Assess Protection Offered by Microencapsulation on Agents with Limited Stability in Biological Fluids

Free 3BP, sCD-3BP and HPCD-3BP were incubated at various concentrations (12.5 μM, 25 μM, 50 μM, 100 μM and 200 μM) with 90 μL of mouse sera at 37° C. for up to 8 hours. Aliquots were collected at 30 minutes, 1 hour, 2 hours, 4 hours and 8 hours and stored at −80° C. until further analyses. Un-degraded biologically active 3BP at each time was then assessed by a cell toxicity assay. Briefly, HCT-116 cells plated at 35-40% confluence in DMEM were treated with a dilution series (in duplicate) of each collected serum sample containing the 3BP formulations. Cell death was measured by imaging cells after 72 hours using the IncuCyte® Live Cell-Analysis System (Essen Bioscience) and using the CellTox™ Green Cytotoxicity Assay (Promega), following the manufacturer's recommendation. Data is presented as percentage of cells alive at each dilution compared to untreated cells as controls in FIG. 2. This provides an estimation of protection offered by microencapsulation in biological fluids.

### Example 2: Demonstrating of Potent Cell Death of sCD-3BP in a Representative Panel of Human Pancreatic Cancer Cell Lines

To assess the ability of sCD-3BP to induce dose-dependent cell death in representative panel of pancreatic cancer lines, sCD-3BP was tested on five pancreatic cancer cell lines: AsPC-1, BxPC-3, CFPAC-1, PSN-1, and Panc02.13. It has been previously demonstrated a robust cytotoxic effect of CD-3BP in MiaPaCa-2 and Suit-2 (Chapiro, J. et al., Clinical cancer research: an official journal of the American Association for Cancer Research 20, 6406-6417, doi:10.1158/1078-0432.ccr-14-1271 (2014)). It has also shown that free 3BP and microencapsulated 3BP displayed similar cytotoxic profile and that CD alone affect cell viability regardless of concentrations (Chapiro, J. et al., Clinical cancer research: an official journal of the American Association for Cancer Research 20, 6406-6417, doi:10.1158/1078-0432.ccr-14-1271 (2014)). To demonstrate broad activity on pancreatic cancer, the cell lines were treated with varying concentrations of sCD-3BP ranging from 0 to 220 uM. All cell lines, barring CFPAC-1, exhibited exquisite sensitivity sCD-3BP, with their IC50s ranging from 27-33 uM (FIGS. 3A and 3B and Table 2). Surprisingly, CFPAC-1 appeared quite insensitive with marginal loss of viability at the highest tested concentration (220 uM).

TABLE 2

| Cell line | IC50 (uM) | MCT-1 (TPM) | Origin |
|---|---|---|---|
| PSN-1 | 28 | 159 | PDAC |
| Panc 02.13 | 30 | 137 | PDAC |
| AsPC-1 | 37 | 65 | PDAC |
| BxPC-3 | 37 | 64 | PDAC |
| Suit-2 | 25-50 | 98 | PDAC |
| MiaPaCa-2 | 27 | 163 | PDAC |
| CFPAC-1 | >200 (not achieved) | 20 | PDAC |
| DLD-1 | 27 | 363 | CRC |
| HCT-116 | 29 | 178 | CRC |
| Colo-205 | >200 (not achieved) | 5 | CRC |
| LS-180 | 26 | 175 | CRC |

### Cell Culture and Viability Assay

The following cell lines were tested in an in vitro assay: MiaPaCa-2, BxPC-3, CFPAC-1, AsPC-1, PSN-1, Panc 02.13 (pancreatic adenocarcinoma lines), DLD-1, HCT-116, Colo-205. Cell lines were cultured using DMEM (for MiaPaCa-2, CFPAC-1, DLD-1and HCT-116), RPMI 1640 (AsPC-1, BxPC-3, Colo-205, PSN-1 and Panc02.13) and IMDM (LS-180) media, all supplemented with 10% fetal bovine serum and 1% penicillin-streptomycin. Cells (3,000-10,000) were seeded in 96-well plates and treated with the indicated amounts of CD-3BP. After treatment, cells were imaged using the IncuCyte® Live Cell-Analysis System (Essen Bioscience) every 8 hours for 96 hours to measure the percentage of cell death for each concentration compared to untreated cells. All in vitro experiments were conducted in triplicates.

### Low Expression of Monocarboxylate Transporter-1 (MCT-1) Confers Resistance to CD-3BP

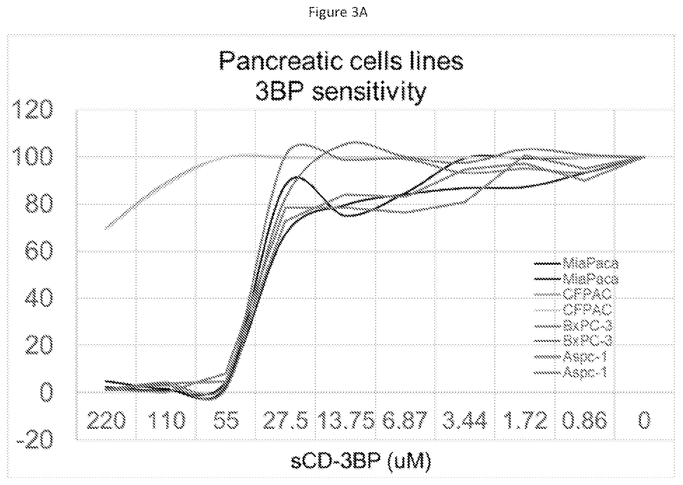

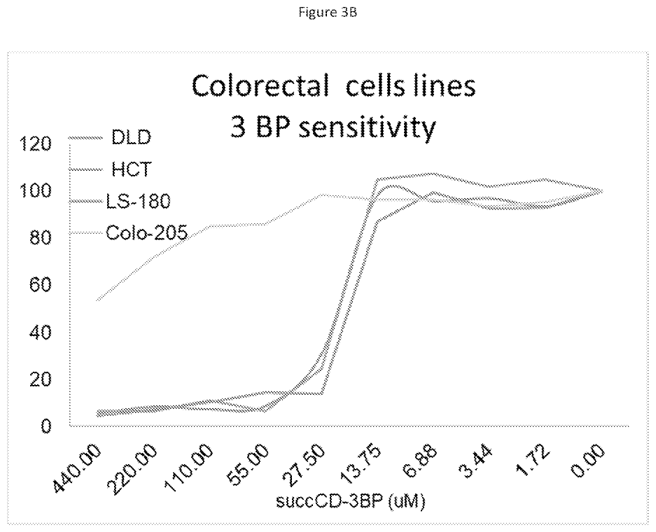

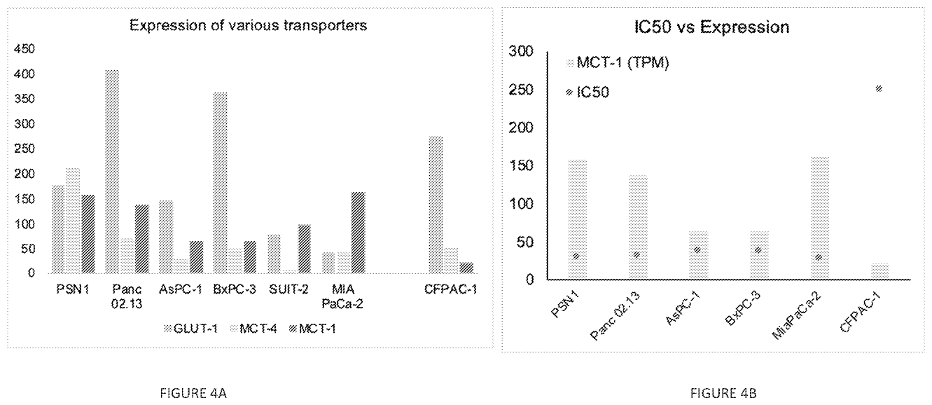

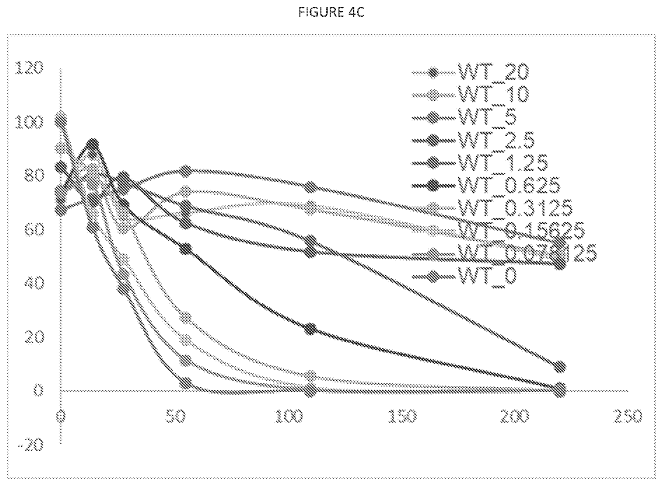

To gain insight into this lack of sensitivity, the expression levels of three cell surface channels, GLUT-1 (Yu, M. et al. The prognostic value of GLUT1 in cancers: a systematic review and meta-analysis. Oncotarget 8, 43356-43367, doi:10.18632/oncotarget.17445 (2017)), MCT-1 and MCT-4 (Hong, C. S. et al. MCT1 Modulates Cancer Cell Pyruvate Export and Growth of Tumors that Co-express MCT1 and MCT4. Cell Reports 14, 1590-1601, doi:10.1016/j.celrep.2016.01.057 (2016)) were compared in all 7 pancreatic cancer cell lines using RNA expression atlas (FIG. 4A). While neither GLUT1 (primarily responsible for glucose uptake and therefore, the FDG-PET positive phenotype) nor MCT-4 expression levels of CFPAC-1 were lower than other cell lines in the panel, expression levels of MCT-1 in CFPAC-1 (22 TPM) was nearly three-fold lower than the next lowest expressor (BxPC-3, 64 TPM) in the cell line panel (FIG. 4B). Since MCT-1 is already implicated as a regulator of cellular uptake of 3BP in breast cancer cell lines (Birsoy, K. et al. MCT1-mediated transport of a toxic molecule is an effective strategy for targeting glycolytic tumors. Nature Genetics 45, 104-108, doi:10.1038/ng.2471 (2013)), testing was conducted to determine if pharmacological blockade with a known inhibitor of MCT-1 (AZD3965) (Noble, R. A. et al. Inhibition of monocarboxyate transporter 1 by AZD3965 as a novel therapeutic approach for diffuse large B-cell lymphoma and Burkitt lymphoma. Haematologica 102, 1247-1257, doi:10.3324/haemato1.2016.163030 (2017)), would protect previously sensitive cells from CD-3BP mediated cell death. MiaPaCa-2 cells co-treated with varying doses of AZD3965 concurrent with CD-3BP showed a clear dose dependent rescue of cells from CD-3BP mediated cytotoxicity (FIG. 4C). Given the importance of this transporter in CD-3BP sensitization this phenomenon was chosen for clarification using a genetic model.

### Example 3: Genetic Deletion of SLC16A1 Abrogates Cell Sensitivity to 3BP

To investigate the relationship between MCT-1 status and 3BP sensitivity in pancreatic cancer cell lines, a targeted deletion of MCT-1 was induced in DLD-1 as well as MiaPaCa-2 cells, the cell line most sensitive to CD-3BP (MCT-1 expression-367 TPM), using CRISPR-Cas9. After selection of six single clones, successful ablation was confirmed using next generation sequencing and validated using IHC. None of the knock-out clones {MCT-1(-)} displayed an aberrant phenotype or behavior compared to the parental cells and exhibited similar viability and growth patterns in vitro.

### Method for Genetic Deletion of SLC (MCT-1) in Human Cancer Cells

The Alt-R CRISPR system (Integrated DNA Technologies, IDT) was used to delete SLC16A1, the gene encoding for the MCT-1 protein, in the DLD-1 and MiaPaCa-2 cell lines. The gRNA sequence was designed using CHOPCHOP v.3 12. Briefly, Alt-R CRISPR Cas9 crRNAs (ACCATGCCATTCAGGCTAGT, IDT; SEQ ID NO:1) and Alt-R CRISPR-Cas9 tracrRNA (1072532, IDT) were re-suspended at 100 μM with Nuclease-Free Duplex Buffer (IDT). The crRNAs and tracrRNA were mixed at a 1:1 molar ratio and denatured for 5 min at 95° C., followed by slow cooling to room temperature to duplex prior to mixing with Cas9 Nuclease (1081059, IDT) at a 1.2:1 molar ratio for 15 min. Forty pmoles of the Cas9 ribonucleoprotein (RNP) containing tracrRNA/MCT-1 crRNA duplex were mixed with 2×10⁵ DLD-1 cells in 20 μL of OptiMEM (31985088, ThermoFisher Scientific). This mixture was loaded into a 0.1 cm cuvette (1652089, Bio Rad) and electroporated at 120 V for 16 ms using an ECM 2001 (Harvard Apparatus). Cells were immediately transferred to complete growth medium and cultured for one week. Upon reaching confluence, polyclonal pools were plated at a density of 0.5 to 2 cells per well in 96-well plates and cultured for 3 weeks. Single colonies were transferred into 2 replica 96-well plates. Genomic DNA was harvested from one of the plates using the Quick-DNA™ 96 Kit (Zymo Research) and PCR amplified using Q5® Hot Start High-Fidelity 2X Master Mix (New England BioLabs). The same process was performed for the MiaPaCa-2 cell line.

### Example 4: Validation of Loss of MCT-1 in edited cells by Next Gen Sequencing (NGS)

Targeted next generation sequencing was performed using SafeSeqS (Isaac Kinde 1, Jian Wu, Nick Papadopoulos, Kenneth W Kinzler, Bert Vogelstein; Proc Natl Acad Sci USA, 2011 Jun. 7; 108(23):9530-5. doi: 10.1073/pnas.1105422108. Epub 2011 May 17) to confirm the mutation status of selected clones as described in Table 3.

TABLE 3

| Clone | Clone | | | | | |
|---|---|---|---|---|---|---|
| no | ID | Chrom | Position | Mutation | Base From | MAF |
| 1 | A6 | chr1 | 113471751 | Indel | CCATGACACTTCGCTGGTGGTGG | 99 |
| | | | | | (SEQ ID NO: 2) | |
| 2 | A4 | chr1 | 113471757 | Indel | CACTT (SEQ ID NO: 3) | 98.9 |
| 3 | B4 | chr1 | 113471757 | Indel | CACTT (SEQ ID NO: 3) | 98.8 |
| 4 | D1 | chr1 | 113471762 | Indel | CGCTGGTGGTG (SEQ ID NO: 4) | 98.6 |
| 5 | C1 | chr1 | 113471762 | Indel | CGCTGGTGGTG (SEQ ID NO: 4) | 98.6 |
| 6 | A2 | chr1 | 113471757/113471760 | Indel | CACTT/T (SEQ ID NO: 4/SEQ ID NO: 5) | 49.8/ |
| | | | | | | 49.9 |

### Effect of MCT-1 Loss on the Sensitivity of Cells to CD-3BP

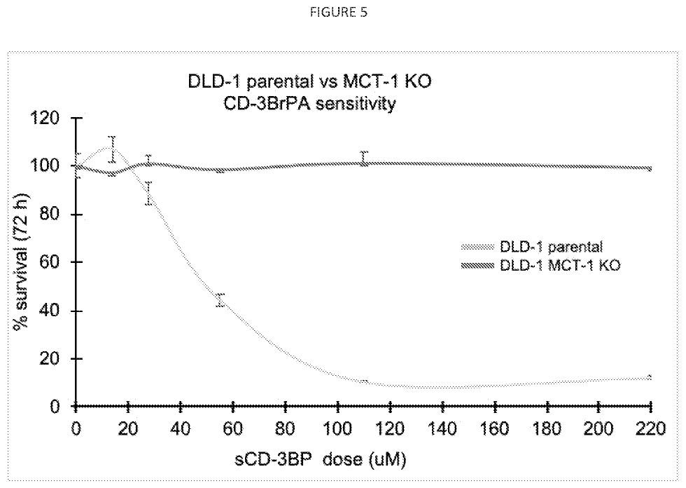

The potency of CD-3BP on a polyclonal mix of NGS validated knock out clones were compared with the parental cells. Treatment of DLD-1 parental cells with various doses of 3BP (0-200 uM) caused dramatic cell death in the parental clones, as expected. Remarkably, DLD-1 MCT-1(-) cells remain completely resistance to 3BP, even at the 200 uM, with no loss of viability (FIG. 5). This was also the case with co-cultured parental MiaPaCa-2 and MiaPaCa-2 MCT-1(-) cells.

### Example 5: Optimization of MCT-1 Immunohistochemistry (IHC) Protocol for Patient Stratification

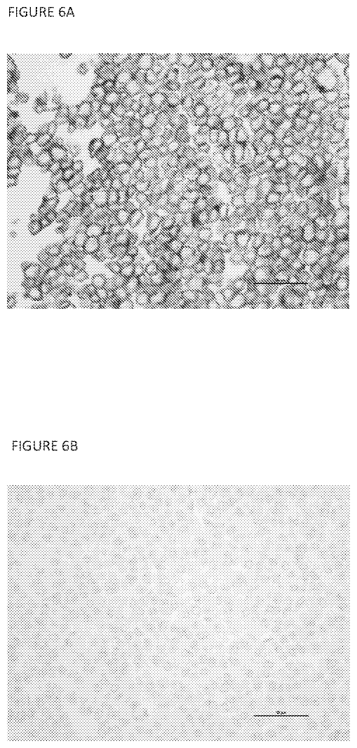

Single clones with evidence at sequencing of successful MCT-1 deletion were collected from the matching second replica plate. Briefly, cells were trypsinized, washed in media to inactivate trypsin and PBS, and then spun at 400 g for 10 m in order to form a plug, which was subsequently fixed in 10% formalin and embedded in paraffin. 4 μm thick sections were cut from formalin-fixed, paraffin embedded blocks and stained for MCT-1. Using DLD-1 parental cells as positive and the MCT-1 (-) clones as negative controls, 5 commercially available antibodies were tested for specificity, one of which demonstrated exquisite specificity as shown in FIG. 6 (FIG. 6A shows MCT-1 staining in DLD-1 Parental and FIG. 6B shows MCT-1 staining in DLD-1 MCT knock-out).

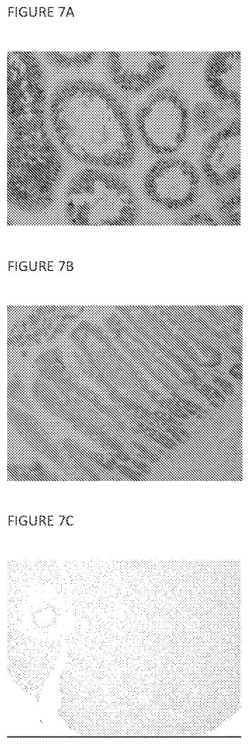

The protocol was further optimized to investigate the pattern of MCT-1 expression in surgically resected pancreatic adenocarcinomas, as well as in several normal tissues. The Human Protein Atlas (http://www.proteinatlas.org) was first queried to determine MCT-1 expression profiles in normal tissues (Uhlén, M. et al. Proteomics. Tissue-based map of the human proteome. Science 347, 1260419, doi:10.1126/science.1260419 (2015)). As result, it was determined that normal gastrointestinal tract and testis display the highest MCT-1 expression levels, whereas normal human pancreas has negligible MCT-1 expression at IHC. Therefore, these tissues were chosen as positive and negative controls, respectively, for optimization of our staining protocol. As expected, colonic mucosa and testis displayed marked staining (FIG. 7A (staining in testes) and FIG. 7B (staining in colon), 20× magnification), whereas we observed no staining in normal pancreatic tissues (FIG. 7C (staining in pancreas), 20× magnification).

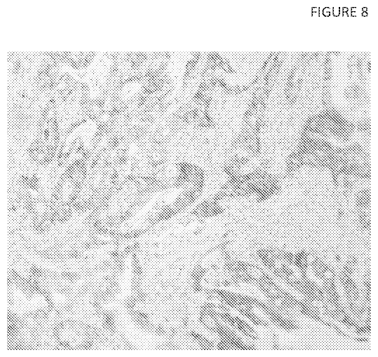

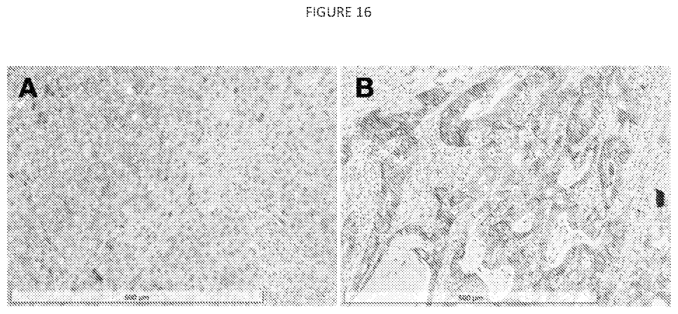

MCT-1 IHC was then evaluated expression in tissue microarrays that included 77 surgically resected pancreatic adenocarcinomas (HPanA150CS03, Biomax U.S.), 75 cholangiocarcinomas (GA802A, Biomax, U.S.), 85 colorectal cancers (BC000110, Biomax, U.S.), and 110 breast cancers (BC08013d and BR10010e Biomax, U.S.). Additionally, another microarray with 20 cases of pancreatic carcinoma (BC001130, Biomax, U.S.) was obtained for further evaluation (FIG. 16). As shown in Table 4, eight out of 77 pancreatic adenocarcinoma cases (10%), demonstrated diffusely high MCT-1 expression (FIG. 8), whereas the remainder exhibited low, patchy or absent expression. Five of 75 cholangiocarcinomas (7%) exhibited high MCT-1 expression. Of 85 colorectal cancer cases, 23 (27%) exhibited high MCT-1 expression, and of 110 breast cancer cases, 16 (15%) exhibited high MCT-1 expression (Table 4). Interestingly, when breast cancer was divided and examined by subtypes, the prevalence of high MCT-1 expression increased to 31% in triple-negative breast cancer (10/32 cases).

TABLE 4
(note that some cores of the original tissue array
were lost or not stained during processing, which
is not uncommon for this type of processing)

| | Cancer | Samples | High | |
|---|---|---|---|---|
| Array | Type | stained | MCT-1 | Percentage |
| HPanA150CS03, Biomax US. | PDAC | 77 | 8 | 10.39% |
| GA802A, Biomax, US | Cholangio | 75 | 5 | 6.67% |
| BC000110, Biomax, US | CRC | 85 | 23 | 27.06% |
| BC08013d and BR10010e, | TNBC | 32 | 10 | 31.25% |
| Biomax, US | | | | |

### MCT-1 Immunohistochemical Staining Protocol

Unstained 4μm sections were cut from formalin-fixed, paraffin embedded blocks.

Immunostaining was performed at the Oncology Tissue Services Core of Johns Hopkins University School of Medicine. Immunolabeling for MCT-1 was performed on formalin-fixed, paraffin embedded sections on a Ventana Discovery Ultra autostainer (Roche Diagnostics). Briefly, following dewaxing and rehydration on board, epitope retrieval was performed using Ventana Ultra CC1 buffer (catalog #6414575001, Roche Diagnostics) at 96° C. for 64 minutes. Primary antibody, anti-MCT-1 (1:optimal dilution; catalog #sc-365501, Lot number D2319, SantaCruz Biotechnology) was applied at 36° C. for 60 minutes. Primary antibodies were detected using an anti-mouse HQ detection system (catalog #7017936001 and 7017782001, Roche Diagnostics) as applicable followed by Chromomap DAB IHC detection kit (catalog #5266645001, Roche Diagnostics), counterstaining with Mayer's hematoxylin, dehydration and mounting. Slides were then dehydrated and mounted permanently for evaluation.

The optimal dilutions for different systems were:
Cell pellet slides (overexpression and knock-out): 1:2000
PDX systems: 1:200
Human colon tissue and TMA: 1:100

### Example 6: In Vitro Evaluation of sCD-3BP

Rapid induction of cell death caused by 3BP:

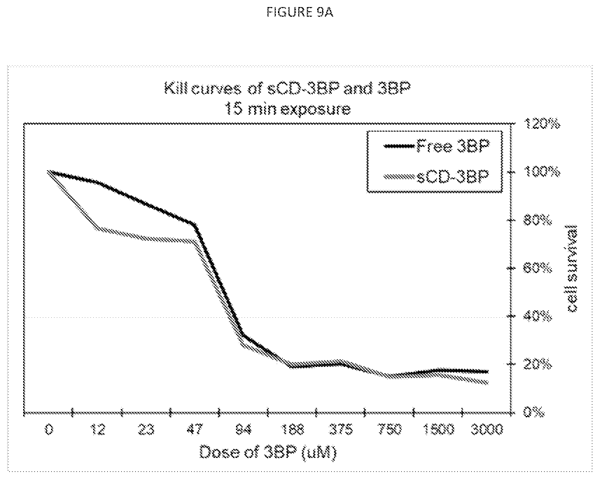

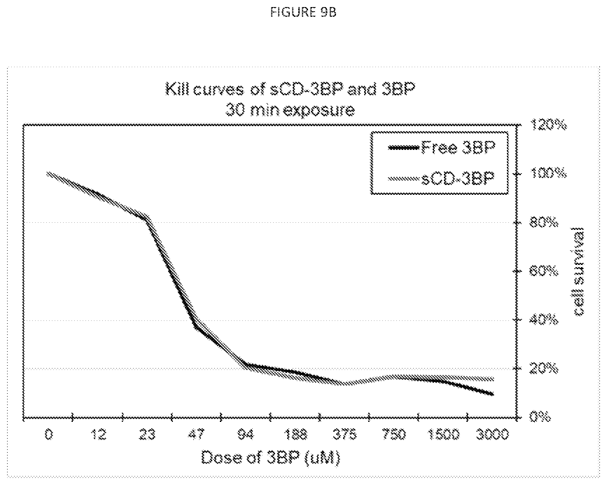

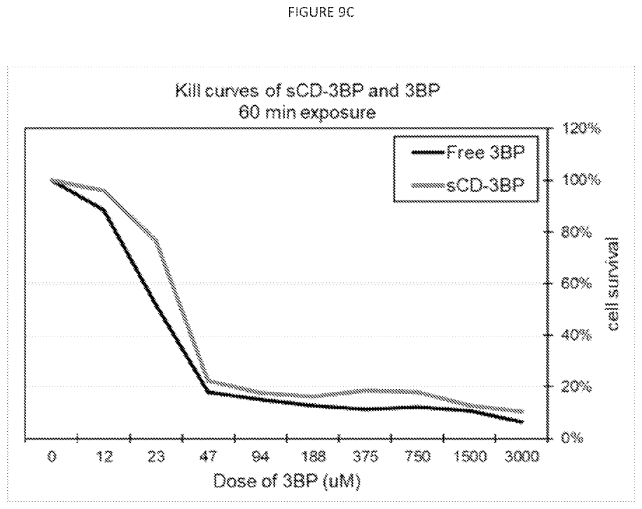

Representative pancreatic cancer cells MiaPaCa-2 (MCT-1: 163 TPM, Expression Atlas) were plated in a 96 well plate at 12,000 cells/well. These cells were then exposed to a 2-fold dilution series of sCD-3BP and free 3BP for different amounts of time (exposure time: 15 minutes, 30 minutes, 1 hour, 2 hours, 4 hours and 8 hours) and their outcome recorded by Incucyte® Live-Cell Analysis Systems, over 36 hours. After the desired timed exposure, cells were washed with PBS and fresh media was added to ensure no more drug persisted in the respective wells. The results indicated that both sCD-3BP and 3BP were equally potent at killing MiaPaCa-2 cells and that there was no loss or delay introduced by caging. Additionally, exposure of MiaPaCa-2 cells to sCD-3BP and 3BP even for a very short time resulted in efficient induction of cell death. The IC₅₀ of both agents was calculated to be 84-90 uM when cells were exposed to the drug for only 15 minutes. A 30 minute exposure resulted in IC₅₀ 30-40 uM and longer exposures did not lower the IC₅₀ dramatically. While treated cells looked initially healthy following drug washout, cell death became discernable 24 hours post exposure. In conclusion, only 15 minutes of exposure to sCD-3BP activates cell death mechanisms that will result in cell clearance within 24 hours without the need of continued exposure to the drug (FIG. 9). This rapid induction of cell death by sCD-3BP is a clear demonstration of the potency and the promise of sCD-3BP as a potential therapeutic strategy in pancreatic cancers.

### Assessment of CD-3BP In Vivo

Encouraged by the in vitro specificity and efficacy demonstrated by sCD-3BP, its performance in MCT-1 high orthotopic models of pancreatic cancer was evaluated. For our first trial, we used Panc 02.13, a pancreatic adenocarcinoma line expressing high levels of MCT-1 (137 TPM-Expression Atlas).

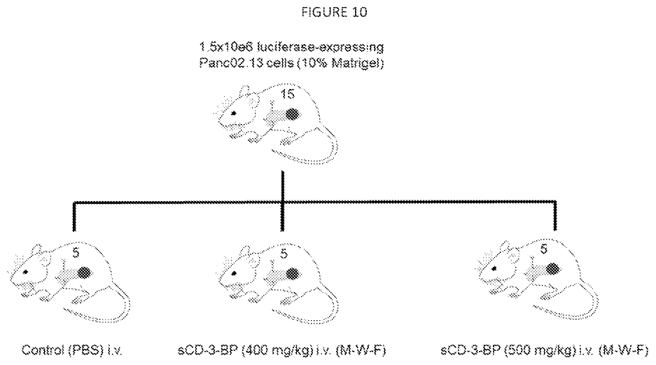

Methods: Panc 02.13 cells were engineered to express firefly luciferase using a luciferase lenti virus from Cellomics Tech, using standard techniques to provide bioluminescence for live cell tracking by IVIS® Optical Imaging. 1.5 million luc-Panc 02.13 cells with 10% Matrigel was implanted in the pancreas of 20 nude mice on day 0. The tumors were allowed to grow for 13 days and evaluated with D-Luciferin Firefly, potassium salt, 1.0 g/vial (PerkinElmer®) by IVIS. Tumor take rate was of 75%. Fifteen animals with similar bioluminescence signal were then randomized into 3 cohorts (Control, 400 mg/kg sCD-3BP, and 500 mg/kg sCD-3BP). On day 16 a second baseline image was recorded and treatment was started on day 17. The animals were treated by an intravenous bolus for 4 weeks with 3 doses/week (Monday, Wednesday, Friday) and bioluminescence was recorded once a week (FIG. 10). At the end of 4 weeks, a final bioluminescence image was taken, the animals in each group were euthanized, and their tumors were evaluated.

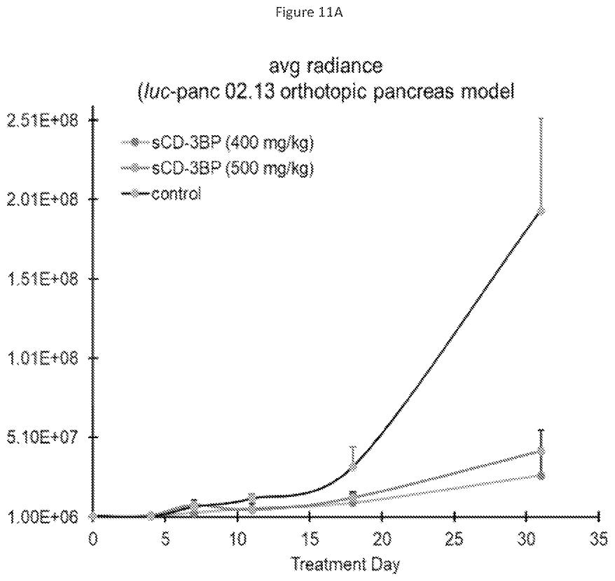

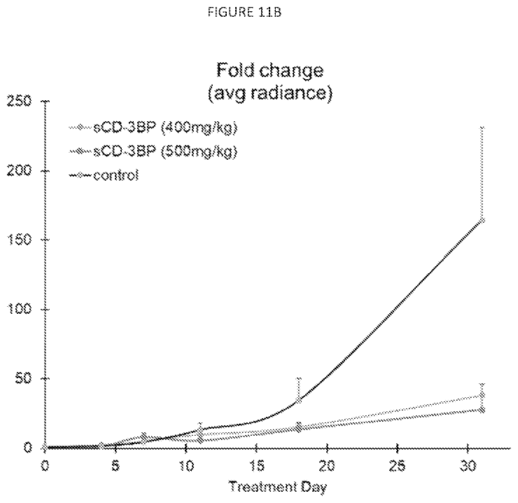

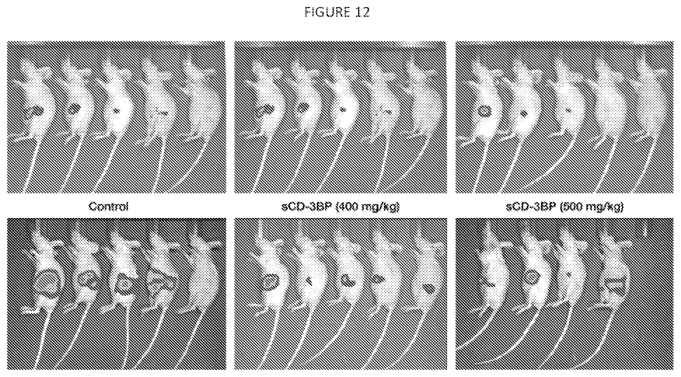

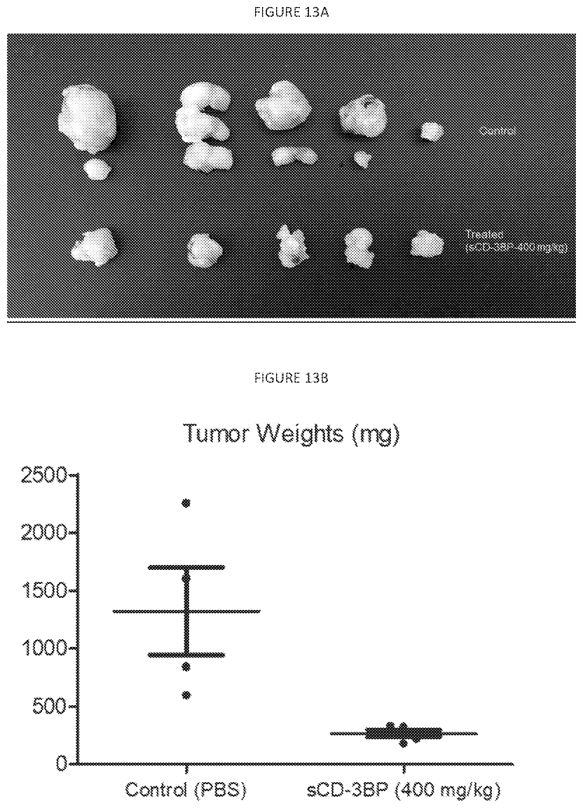

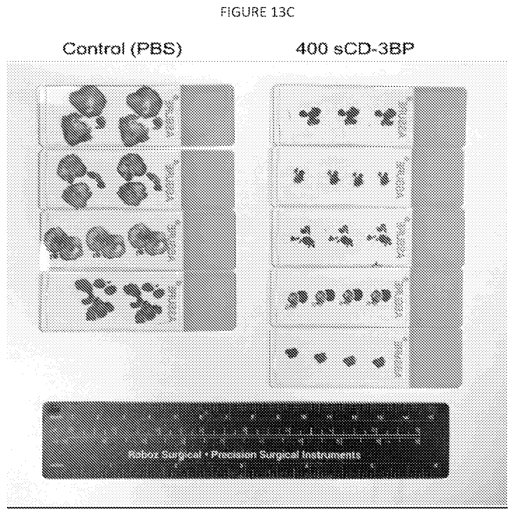

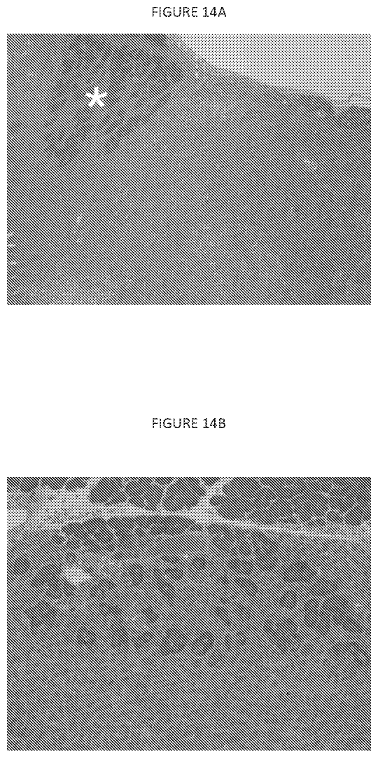

Results: Treatment of orthotopic implanted luc-Panc02.13 bearing mice with 400 mg/kg or 500 mg/kg doses of sCD-3BP resulted in dramatic slowing down of tumor progression. Administered intravenously, sCD-3BP was very well tolerated at 400 mg/kg doses with no major weight loss. Strong anticancer effects were observed at both doses with effects noticeable as early as 14 days post treatment initiation. Animals in the vehicle treated control group demonstrated a 140-200 fold increase in signal from baseline (FIGS. 11 and 12), while animals in both treatment cohorts showed minimal progression with a 5-6 fold lower signal than the control arm. At the end of the treatment animals were euthanized, and underwent a necropsy. Residual tumors and major organs were harvested for further analyses. The effect of sCD-3BP treatment was quite visible upon inspection of the tumors. Residual tumors from the animals in sCD-3BP treated group were significantly smaller in size, weighed less, and when sectioned had an appreciably smaller diameter (FIGS. 13 and 14).

### Example 7: CD-3BP Reduces Tumor Burden in Orthotopically Implanted Human Patient Derived Xenografts with Moderate-High MCT-1 Expression (PDx in NSG)

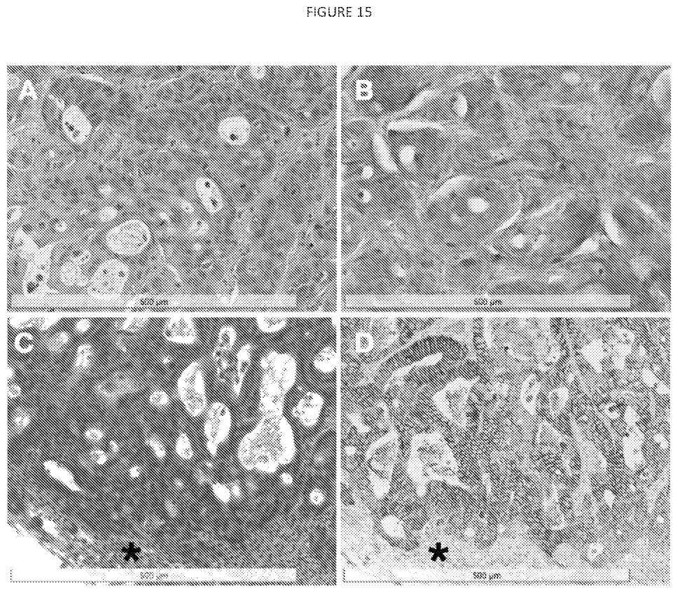

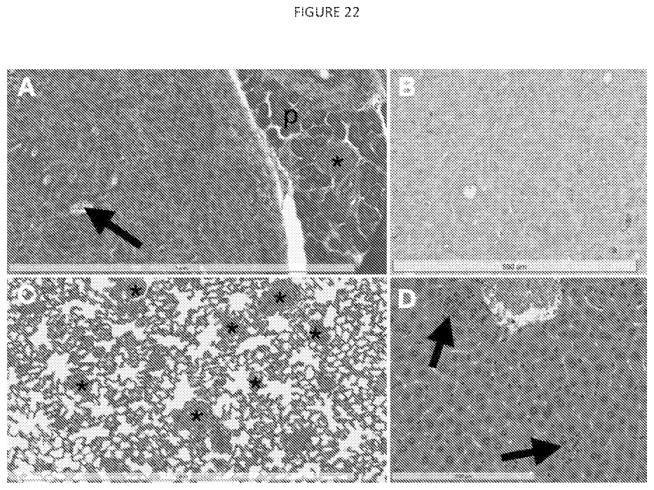

In order to rigorously evaluate the potential of sCD-3BP as a therapeutic, more complex models of human pancreatic cancer were explored. Patient derived xenografts (PDx) generated by transplanting fresh tumor tissue collected from patients with pancreatic ductal adenocarcinoma (PDAC) under the skin of immunosuppressed mice represent a significantly better model for preclinical assessment of drugs than cancer cell line xenograft models (Izumchenko E, et al. Patient-derived xenografts effectively capture responses to oncology therapy in a heterogeneous cohort of patients with solid tumors. Ann Oncol. 2017; 28(10):2595-2605. doi: 10.1093/annonc/mdx416). It is well established that non-orthotopic or cell-line derived models do not fully recapitulate the characteristics of the primary disease, their response to therapies, or their metastatic potential (Martinez-Garcia R, Juan D, Rausell A, et al. Transcriptional dissection of pancreatic tumors engrafted in mice. Genome Med. 2014; 6(4):27. doi:10.1186/gm544). Despite that, and given the complexity of obtaining and maintaining PDx, few groups utilize PDx in their PDAC research. Orthotopic implanted PDx models were created that mimicked the local and systemic aspects of human disease more faithfully. To perform these experiments two PDx samples demonstrating moderate to high levels of MCT-1, out of 12 available (Table 5), with full genomic and transcriptomic characterization were acquired from Jackson labs and their MCT-1 status was confirmed by IHC (FIG. 15). Interestingly, those models have specific histopathological characteristics typical of some PDAC including dispersed and invasive growth with pancreas invasion, pancreatic inflammation, ductal formation, local metastasis to the peritoneal wall, and distant metastasis to the liver and lung (FIG. 22).

TABLE 5

| | Primary | Specimen | | Clinical | Tumor | | | | | |
|---|---|---|---|---|---|---|---|---|---|---|
| Model ID | Tumor Site | Site | Tumor Type | Diagnosis | Stage | Sex | Age | Race | Gene | fpkm |
| TM01098 | Pancreas | Liver | Metastatic | pancreatic | AJCC IV | Male | 76 | White | SLC16A1 | 70.48 |
| | | | | carcinoma | | | | | | |
| TM00920 | Pancreas | Pancreas | Primary | mucinous | AJCC IIB | Female | 65 | White | SLC16A1 | 46.36 |
| | | | | adenocarcinoma | | | | | | |
| J000096053 | Pancreas | Lung | Metastatic | pancreatic | AJCC IV | Male | 64 | White | SLC16A1 | 32.32 |
| | | | | adenocarcinoma | | | | | | |
| J000096053 | Pancreas | Lung | Metastatic | pancreatic | AJCC IV | Male | 64 | White | SLC16A1 | 29.94 |
| | | | | adenocarcinoma | | | | | | |
| J000096053 | Pancreas | Lung | Metastatic | pancreatic | AJCC IV | Male | 64 | White | SLC16A1 | 28.03 |
| | | | | adenocarcinoma | | | | | | |
| TM01007 | Pancreas | Pancreas | Primary | adenocarcinoma | Not | Female | 81 | White | SLC16A1 | 27.51 |
| | | | | | Specified | | | | | |
| J000077973 | Pancreas | Pancreas | Primary | adenosquamous | Not | Female | 68 | White | SLC16A1 | 26.7 |
| | | | | carcinoma | Specified | | | | | |
| TM01212 | Pancreas | Pancreas | Not | pancreas | Not | Male | | NA | SLC16A1 | 25.12 |
| | | | Specified | adenocarcinoma | Specified | | | | | |
| TM01125 | Pancreas | Pancreas | Recurrent/ | neuroendocrine | AJCC IV | Male | 79 | White | SLC16A1 | 25.03 |
| | | | Relapse | carcinoma | | | | | | |
| TM01007 | Pancreas | Pancreas | Primary | adenocarcinoma | Not | Female | 81 | White | SLC16A1 | 23.06 |
| | | | | | Specified | | | | | |
| J000077960 | Pancreas | Pancreas | Primary | pancreatic ductal | AJCC IV | Female | 68 | White | SLC16A1 | 18.15 |
| | | | | adenocarcinoma | | | | | | |

Sample TM01212 was a pancreas adenocarcinoma sample of unspecified stage obtained from a male of unspecified age. After propagating as a subcutaneous xenograft in NSG (NOD.Cg-Prkdc^{scid}II2rg^{tm1Wjl}/SzJ) mice, these tumors were excised, sectioned into defined pieces and then transplanted into the pancreas of NSG mice. In the pancreas these tumors grew more rapidly, showed significant invasion of the pancreas, metastasized to lung and liver in the hosts just like in patients with advanced pancreatic cancers.

In absence of bioluminescence signal, a non-invasive ultrasound technique was developed to routinely track the growth and response to sCD-3BP of these internal tumors akin to how such a tumor maybe tracked in real patients. Having solved the technical challenges associated with ultrasound measurements on PDACs in mice, the response of sCD-3BP in this unique model using TM01212 was evaluated.

### Method for Ultrasound Measurements of Orthotopic PDx Tumors

The left flanks of the mice were shaved in a standard fashion. A Vevo2100 ultrasound system was used for in vivo ultrasound visualization of orthotopically implanted PDAC. After anesthesia induction with 2% isoflurane in the induction chamber, the mice were injected with 2 mL of sterile saline intraperitoneally. Then, the mice were placed in the right recumbent position in a heated pad for imaging of the left flank. Continuous anesthesia was provided with a nose cone during the entire imaging. A layer of ultrasound gel was used over the left flank to allow visualization. The imaging protocol was standardized to obtain imaging (FIG. 17) and measurements in the same orientation for each mice. The tumor was observed after identification of normal intrabdominal structures such as the spleen and stomach. Trans-axial and longitudinal images and videos were obtained for measurements of each tumor (Stephen A. Sastral and Kenneth P. Olive. Quantification of Murine Pancreatic Tumors by High Resolution Ultrasound. Methods Mol Biol. 2013; 980: doi:10.1007/978-1-62703-287-2_13).

### Method for Pancreatic Transplants

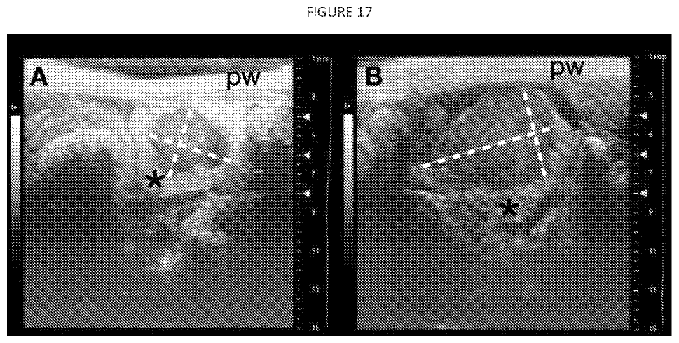

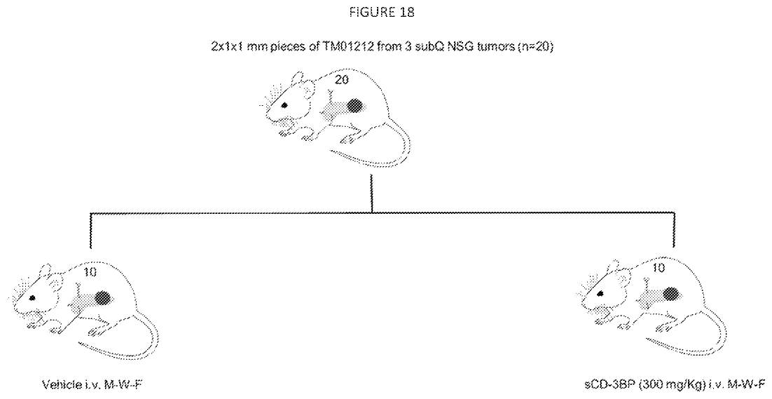

20 NSG mice were transplanted with 2×1×1 mm sized pieces of TM01212 tumors harvested from 3 hosts bearing subcutaneous versions of these. The first ultrasound measurement was recorded on day 13 post implantation. Once it was confirmed that the implanted TM01212 tumors were efficiently transplanted (take rate 90%), these animals were randomized into 2 cohorts (n=10). The control group was infused with the vehicle (270 mg/kg sCD) via intravenous tail vein bolus every Monday, Wednesday, and Friday while the treatment group was treated with 300 mg/kg sCD-3BP at the same schedule (FIG. 18). Ultrasound measurements were taken every Thursday. The host animals were subjected to this regimen for 4 weeks and euthanized on day 30 post treatment initiation. Tumor burden in the pancreas as well at distant sites was assessed by necropsy.

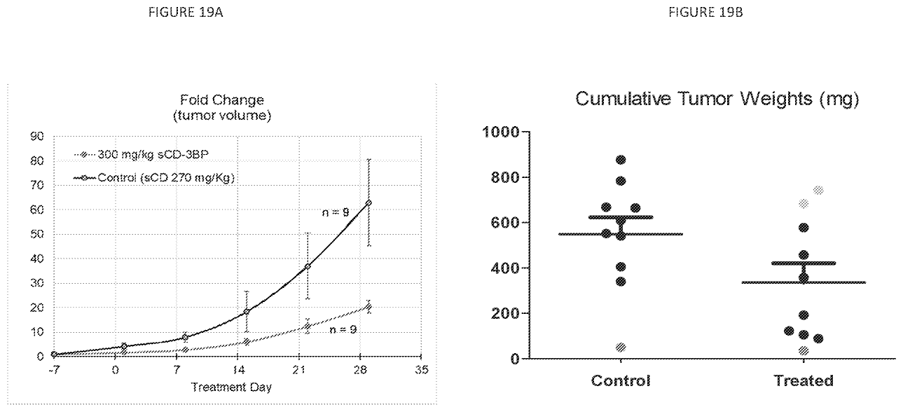

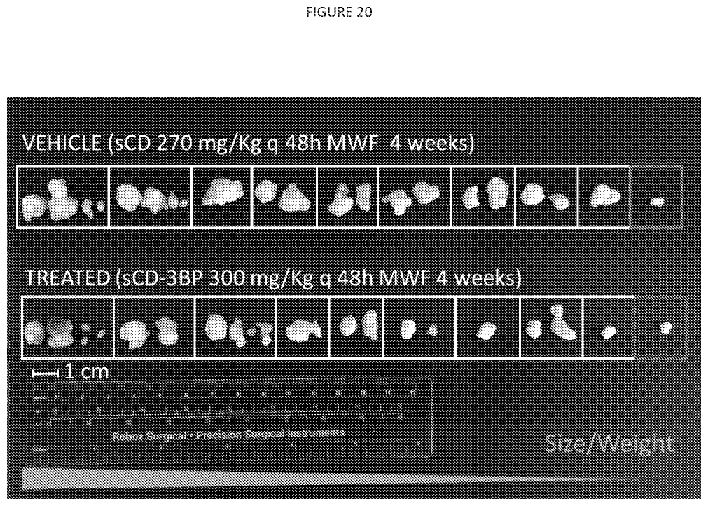

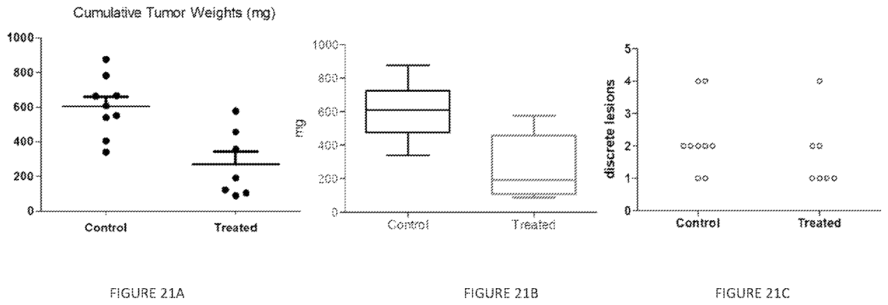

Results: TM01212 implanted in the pancreas of host mice exhibited strikingly similar clinical features characteristic of human PDACs. Compared to subcutaneous implants of TM01212, the orthotopically implanted model produced metastatic lesions to lung and liver, significant invasion into pancreas, and development of duct like structures within the tumor. However, this strain of mice (NSG) were able to tolerate a slightly reduced dose of 300 mg/kg sCD-3BP when treated every Monday, Wednesday, Friday for 4 weeks. In addition, in these experiments, scarring/scab formation was noticed due to repeated punctures in the tail of 2 animals in the treatment group. This resulted in 2 animals being administered a lower cumulative dose (70% compared to cohort mates) that the rest of the cohort. Despite a lower administered dose in this model, sCD-3BP produced a robust anti-tumor response, slowing down the progression of these aggressive tumors significantly (FIG. 19A). Upon euthanasia and follow-up necropsy, sCD-3BP treated animals had a lower metastatic burden compared to their vehicle treated counterparts (FIG. 19B). Treatment of TM01212 with sCD-3BP also resulted in fewer metastatic lesions. It was noted that the 2 animals which did not receive the full cumulative dose in the treated group had the highest tumor burden in that cohort thus further correlating the response with the delivered dose of sCD-3BP (FIGS. 20 and 21).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

What is claimed is:

1. A composition comprising at least one β-cyclodextrin and at least one cytotoxic receptor binding small-molecule, wherein the β-cyclodextrin encapsulates the cytotoxic receptor binding small-molecule.

2. The composition of claim 1, wherein at least one α-D-glucopyranoside unit of the cyclodextrin has at least one hydroxyl chemical group replaced with an ionizable chemical group resulting in a negative charge and wherein the cyclodextrin encapsulates the at least one cytotoxic receptor binding small-molecule.

3. The composition of claim 2, wherein at least one C2, C3, and C6 hydroxyl chemical groups of at least one α-D-glucopyranoside unit of the cyclodextrin are replaced with ionizable chemical groups.

4. The composition of claim 3, wherein the at least one α-D-glucopyranoside unit of the cyclodextrin is selected from the group consisting of two, three, four, five, six, seven, eight, and all α-D-glucopyranoside units of the cyclodextrin.

5. The composition of claim 4, wherein the ionizable chemical group is the same at all replaced positions.

6. The composition of claim 5, wherein the ionizable chemical group is a weakly basic functional group or a weakly acidic functional group.

7. The composition of claim 6, wherein the weakly basic or weakly acidic functional groups are selected from the group consisting of amino, ethylene diamino, dimethyl ethylene diamino, dimethyl anilino, dimethyl naphthylamino, succinyl, carboxyl, sulfonyl, and sulphate functional groups.

8. The composition of claim 1, wherein the composition is a liquid or solid pharmaceutical formulation.

9. The composition of claim 1, wherein the β-cyclodextrin is selected from the group consisting of 6′ modified β-cyclodextrin, 6′ mono-succinyl β-cyclodextrin, hydroxypropyl-β-cyclodextrin, and succinyl-β-cyclodextrin.

10. The composition of claim 1, wherein the cytotoxic receptor binding small-molecule is haloacetate, halopyruvate, halolactate, halopropionate or halobutyrate or combinations thereof.

11. The composition of claim 1, wherein the composition is formulated for systemic administration.

12. A kit comprising a composition of claim 1, and instructions for use.

13. A method of treating a subject having a cancer comprising administering to the subject a therapeutically effective amount of a composition of claim 1.

14. The method of claim 13, wherein the composition is administered systemically.

15. The method of claim 14, wherein the systemic administration is selected from the group consisting of oral, intravenous, intrathecal, intraperitoneal, subcutaneous, and intramuscular administration.

16. The method of claim 14, wherein the subject is treated with at least one additional anti-cancer therapy.

17. The method of claim 14, wherein the cancer is expressing MCT-1.

18. The method of claim 14, wherein the cancer is selected from the group consisting of liver cancer, pancreatic cancer, bile duct cancer, colorectal cancer, mesothelioma, leukemias, germ cell tumors, glioma, lung cancer, ovarian cancer, prostate cancer, head and neck cancers, melanoma, stomach cancer, bone cancer, renal cancer, bladder cancer, and breast cancer.

19. The method of claim 18, wherein the cancer is breast cancer.

20. The method of claim 19, wherein the breast cancer is triple negative breast cancer.

21. The method of claim 14, wherein the subject is a mammal.

22. The method of claim 21, wherein the mammal is a human.

23. A method for assessing the stability of a composition comprising at least one agent encapsulated in a β-cyclodextrin, the method comprising the steps of:
a. providing at least one β-cyclodextrin and at least one agent, wherein the at least one agent is encapsulated in the at least one β-cyclodextrin to provide at least one β-cyclodextrin encapsulated agent composition;
b. assessing the composition for presence or absence of 3-bromopyruvate using a cell toxicity assay; and
c. determining the stability of the composition.

24. The method of claim 23, wherein the composition is incubated in sera for at least 30 minutes prior to performing the cell toxicity assay.

25. The method of claim 23, wherein the agent is at least one cytotoxic receptor binding small-molecule.


SEQUENCE LISTING (5 sequences)

SEQ ID NO: 1 - protein, 23 aa, Artificial Sequence
  Synthetic
ACCATGCCATTCAGGCTAGTIDT

SEQ ID NO: 2 - protein, 23 aa, Artificial Sequence
  Synthetic
CCATGACACTTCGCTGGTGGTGG

SEQ ID NO: 3 - protein, 5 aa, Artificial Sequence
  Synthetic
CACTT

SEQ ID NO: 4 - protein, 11 aa, Artificial Sequence
  Synthetic
CGCTGGTGGTG

SEQ ID NO: 5 - protein, 6 aa, Artificial Sequence
  Synthetic
CACTTT